(19) United States
(12) Patent Application Publication
(10) Pub. No.: US 2010/0042117 A1
(43) Pub. Date: Feb. 18, 2010

(54) SUTURE PASSER

(76) Inventors: Andrew C. Kim, Temecula, CA (US); Ronald Van Elderen, Oceanside, CA (US); James W. Chang, Loma Linda, CA (US)

Correspondence Address: James W. Chang, P.O.Box 1497, Loma Linda, CA, 92354, US

(21) Appl. No.: 12/539,207

(22) Filed: Aug. 11, 2009

Related U.S. Application Data
(60) Provisional application No. 61/136,119, filed on Aug. 13, 2008.

(51) Int. Cl. A61B 17/10

(52) U.S. Cl. 606/148

(57) ABSTRACT

Disclosed are devices and methods relating to a suture passer that passes suture efficiently via motion of fluid in and out of the suture passer. In certain embodiments, the suture passer includes a syringe-like barrel and a plunger for drawing and expelling fluid from the barrel. A suture can be disposed within the barrel, and one end of the suture can be releasably secured to a portion of the plunger. In certain embodiments, the suture end can be releasably secured to a tip portion of the plunger. Various examples of suture releasing mechanisms and actuations of such mechanisms are disclosed. Also disclosed are examples of how such releasably secured sutures can be assembled and packaged into pre-loaded suture passer devices. Also disclosed are examples of suturing operations that can be achieved easier and quicker by use of such suture passer devices.

### RELATED APPLICATION

This application claims priority benefit of U.S. Provisional Patent Application No. 61/136,119 filed Aug. 13, 2008, titled “HYDRODYNAMIC SUTURE PASSER,” which is incorporated herein by reference in its entirety.

### BACKGROUND

1. Field

The present disclosure generally relates to the field of surgical instruments, and more particularly, to devices and methods for passing sutures during surgical procedures.

2. Description of the Related Art

In many surgical procedures, a need to immobilize a tissue arises. For example, a torn ligament or tendon may need to be re-attached to a bone. Such re-attachment can be achieved by using a suture to hold down the tissue to a desired location such as a bone. The suture can be threaded through one or more locations on the tissue so as to form a desired pattern.

Although such desired suture patterns may appear to be simple in isolation, the actual threading of the suture can be difficult and tedious due to the operating site being difficult to access. Further, the operating site can be small, thereby making manipulation of suture and suturing instruments difficult. Consequently, use of conventional suturing instruments for certain suturing procedures requires inefficient manipulations of the suture and the instruments.

### SUMMARY

The present disclosure relates to a syringe assembly having a barrel that defines a chamber and a conduit having a reduced dimension. The syringe assembly further includes a plunger having a suture retaining mechanism. The syringe assembly further includes a suture substantially positioned in the chamber, with one end of the suture held releasably by the suture retaining mechanism.

In certain embodiments, the barrel includes a needle-coupling mechanism at or about the conduit so as to allow flow of liquid through a needle coupled to the barrel as the liquid is expelled from or drawn into the chamber by action of the plunger. In certain embodiments, other end of the suture is positioned at or about the conduit so as to allow the other end of the suture to enter the needle. In certain embodiments, the syringe assembly further includes a conduit feature disposed on or about the conduit and configured to inhibit the other end of the suture from being drawn into the chamber.

In certain embodiments, the suture retaining mechanism includes a deformable tip of the plunger. The deformable tip can include a feature formed at its front, with the front facing the chamber. The feature can be configured to retain the one end of the suture in a closed configuration and release the suture in an open configuration. In certain embodiments, the plunger further includes an actuator mechanism that transmits a force to the deformable tip so as to facilitate transition of the feature from the closed configuration to the open configuration. In certain embodiments, the actuator mechanism includes an actuator rod that provides a pushing force to the deformable tip from rear of the deformable tip so as to deform an area at or about the feature sufficiently to yield the transition. In certain embodiments, the actuator mechanism further includes a hollow rod coupled to the deformable tip, with the hollow rod defining a bore dimensioned to receive the actuator rod and allow axial motion of the actuator rod relative to the hollow rod so as to provide the pushing force to the deformable tip.

In certain embodiments, the feature includes one or more slits formed on the deformable tip and dimensioned to squeeze and retain the one end of the suture in the closed configuration and spread and release the suture in the open configuration. In certain embodiments, the slit is dimensioned such that the rubber tip retains its fluid sealing property when the slit is in either the closed or open configuration.

In certain embodiments, the suture can be stored in a selected configuration relative to a feature associated with the plunger. In certain embodiments, the feature includes a plunger tip having a reduced diameter portion such that the suture is wound about the reduced diameter portion. In certain embodiments, the feature includes a recess defined by a tip of the plunger such that the suture is in a coiled configuration substantially within the recess.

In certain embodiments, the suture retaining mechanism includes a jaw assembly disposed at a tip of the plunger. The jaw assembly can be configured to retain the suture in a closed position, and release the suture in an open position. Opening of the jaw assembly can be induced by an interaction of the jaw assembly with a feature associated with the barrel.

In certain embodiments, a kit can include the aforementioned syringe assembly, and a sterile packaging for the syringe assembly. In certain embodiments, the syringe assembly can be packaged substantially dry. In certain embodiments, the syringe assembly can be packaged pre-loaded with fluid in the chamber.

The present disclosure also relates to a device having a barrel having proximal and distal ends, and defining an inner volume having a first cross-sectional area and extending axially between the proximal and distal ends. The distal end of the barrel defines an opening having a second cross-sectional area that is less than the first cross-sectional area, and the opening is configured to allow expelling and drawing of fluid from and into the inner volume through the opening. The device further includes a plunger having proximal and distal ends, with the distal end of the plunger configured to fit in the inner volume and be movable axially so as to decrease and increase the inner volume to thereby facilitate the expelling and drawing of the fluid. The device further includes a suture holding mechanism disposed on or about the distal end of the plunger and configured to releasably hold an end of a suture.

In certain embodiments, the device further includes an actuator mechanism configured to release the end of the suture from the suture holding mechanism. In certain embodiments, the actuator mechanism includes one or more parts disposed within the plunger, where the one or more parts are movable relative to the distal end of the plunger. In certain embodiments, the actuator mechanism induces the suture release by an interaction between the plunger and the barrel.

In certain embodiments, the suture holding mechanism includes one or more features formed on a tip disposed at the distal end of the plunger. In certain embodiments, the suture holding mechanism includes one or more parts secured to a tip disposed at the distal end of the plunger.

The present disclosure also relates to a method, for performing a suturing operation. The method includes steps of (a) passing a needle from a first side to a second side of a tissue; (b) expelling a first end of a suture from the needle such that the first end is at the second side; (c) performing one or more cycles of: (i) withdrawing the needle back to the first side such that a part of suture that came out of the needle just prior to the withdrawing remains substantially at the second side; (ii) moving the needle to a different location on the tissue; and (iii) passing the needle from the first side to the second side at the different location such that a part of the suture outside of the needle follows the needle from the first side to the second side; and (d) expelling a second end of the suture from the needle such that the second end is at the second side.

In certain implementations, the expelling of the first and second ends of the suture are achieved by expelling of fluid through the needle. In certain implementations, the expelling of fluid is achieved by a suture passer coupled to the needle, with the suture passer having a chamber defined by a barrel and a plunger, such that the fluid is expelled from the chamber, and at least some of the suture is in the chamber prior to the step (d) such that the second end is releasably retained by the plunger.

In certain implementations, the step (c) is performed once so as to form a single mattress suture pattern. In certain implementations, the step (c) is performed twice so as to form a double mattress suture pattern by passing the first and second ends at second side of the tissue through an intermediate loop of suture formed on second side of the tissue and tightening the first and second ends.

The present disclosure also relates to one or more other features related to suture passer devices and associated methods as described herein.

### BRIEF DESCRIPTION OF THE DRAWINGS

FIG. 1 shows an example suture passer device in communication with a volume of fluid via a needle, showing that flow speed inside the needle is generally higher due to Venturi effect resulting from relatively small lateral dimension of the inside of the needle;

FIGS. 2A and 2B show how the Venturi effect of FIG. 1 can facilitate effective drawing in and expelling of a suture into and from the suture passer device;

FIG. 3 shows that in certain embodiments, the suture passer device can be configured with one or more features that facilitate easier use;

FIGS. 4A-4D show an example sequence of operation of the suture passer device, where one end of a suture is initially retained releasably within the suture passer, and released when desired;

FIGS. 5A and 5B show end and side views of an example plunger having a suture securing mechanism that can be defined by its tip to allow releasable securing of the suture, where the securing mechanism is in a configuration to secure the suture;

FIGS. 6A and 6B show the example plunger of FIGS. 5A and 5B, where the securing mechanism is in a configuration to release the suture;

FIGS. 7A and 7B show end and side views of an example plunger having a suture securing mechanism disposed at or about its tip to allow releasable securing of the suture, where the securing mechanism is in a configuration to secure the suture;

FIGS. 8A and 8B show the example plunger of FIGS. 7A and 7B, where the securing mechanism is in a configuration to release the suture;

FIG. 9 shows that in certain embodiments, activation of the suture securing mechanism to release the suture can be achieved by a mechanism that is part of the plunger;

FIG. 10 shows that in certain embodiments, activation of the suture securing mechanism to release the suture can be achieved at least in part by an interaction between the plunger and a barrel of the suture passer device;

FIGS. 11A and 11B show that in certain embodiments, the suture passer device can include a suture securing mechanism that can be defined by a plunger tip, and suture release actuation of such mechanism can be achieved by a mechanism that is part of the plunger;

FIGS. 12A-12D show various cross-sectional views indicated on the suture passer device of FIG. 11A;

FIGS. 13A-13C show that in certain embodiments, the suture passer device can include a suture securing mechanism that can be defined by the plunger tip, and suture release actuation of such mechanism can be achieved by a spring-loaded mechanism that is part of the plunger;

FIGS. 14A and 14B show that in certain embodiments, the suture passer device can include a suture securing mechanism that can be positioned at or about the plunger tip, and suture release actuation of such mechanism can be achieved by a mechanism that is part of the plunger;

FIGS. 15A and 15B show that in certain embodiments, the suture passer device can include a suture securing mechanism that can be defined by the plunger tip, and suture release actuation of such mechanism can be facilitated via an interaction of the plunger with one or more features associated with a barrel of the suture passer device;

FIGS. 16A and 16B show end views of example closed and open configurations of the plunger tip of FIGS. 15A and 15B;

FIGS. 17A and 17B show that in certain embodiments, the suture passer device can include a suture securing mechanism that can be positioned at or about the plunger tip, and suture release actuation of such mechanism can be facilitated via an interaction of such mechanism with one or more features associated with the barrel;

FIGS. 18A and 18B show that in certain embodiments, suture can be spooled on the outside of a structure at the tip of a plunger, and release of such suture can be achieved by an actuator that is part of the plunger;

FIGS. 19A-19C show that in certain embodiments, suture can be spooled on the outside of a structure at the tip of a plunger, and release of such suture can be achieved by an interaction between the plunger and one or more features associated with a barrel of the suture passer device;

FIGS. 20A and 20B show that in certain embodiments, suture can be spooled on the inside of a recess formed at the tip of a plunger;

FIGS. 21A and 21B show another example configuration where suture can be spooled on the inside of a recess formed at the tip of a plunger;

FIGS. 22A-22D show that in certain embodiments, one or more features associated with a plunger and/or barrel can provide an indication—such as tactile feedback—that can facilitate timing of suture release;

FIGS. 23A-23E show non-limiting examples of how suture can be positioned in the suture passer device so as to facilitate easy preparation and use;

FIG. 24 shows an example of how the suture passer device can be used to perform suturing operation on a tissue inside a body;

FIGS. 25A-25C show an example of a suture being passed from one side of a tissue to the other side;

FIGS. 26A-26C show an example of a suture pattern that can be achieved based on the suture configuration of FIGS. 25A-25C;

FIG. 27 shows an example of a mattress suture pattern that can be achieved by use of the suture passer device;

FIGS. 28A and 28B show an example of a double mattress suture pattern that can be achieved by use of the suture passer device;

FIGS. 29A and 29B show an example of a more complex suture pattern such as a modified Mason-Allen pattern;

FIG. 30 shows an example steps that can be implemented to achieve the example Mason-Allen pattern of FIGS. 29A and 29B;

FIGS. 31A and 31B show examples of additional ergonomic features that can be incorporated into the suture passer device so as to facilitate manipulation of the device; and

FIGS. 32A and 32B show side and isometric views of another example of additional ergonomic features that can be provided to the suture passer device so as to facilitate manipulation of the device.

These and other aspects, advantages, and novel features of the present teachings will become apparent upon reading the following detailed description and upon reference to the accompanying drawings. In the drawings, similar elements have similar reference numerals.

### DETAILED DESCRIPTION OF CERTAIN EMBODIMENTS

The present disclosure generally relates to the field of surgical instruments, and more particularly, to devices and methods for passing sutures during surgical procedures. In many surgeries, various suturing procedures may need to be performed in locations that are relatively difficult to access and/or work in. In such surgical procedures, use of hydrodynamic suture passers can overcome or mitigate at least some of such difficulties. Additional details about such suture passers can be obtained from, for example, U.S. patent application Ser. No. 10/614,653 titled “Hydrodynamic Suture Passer,” filed Jul. 7, 2003, and U.S. patent application Ser. No. 10/883,742 titled “Hydrodynamic Suture Passer,” filed Jul. 6, 2004, each of which is incorporated herein by reference in its entirety.

As described herein, one or more features of the present disclosure can facilitate easier suturing procedures involving manipulation of sutures via fluid movements. It will be understood that such one or more features of the present disclosure can be applied in surgical procedures in human or non-human animal subjects. Such subjects can be living or non-living subjects. In the context of living subjects, such surgical procedures can include orthopaedic surgical procedures such as arthroscopic procedures. Arthroscopic procedures are commonly performed on or about the knee and shoulder joints. Such procedures can also be performed on joints associated with wrists, elbows, ankles and hips. These are some non-limiting examples of procedures where one or more features of the present disclosure can be used in an advantageous manner.

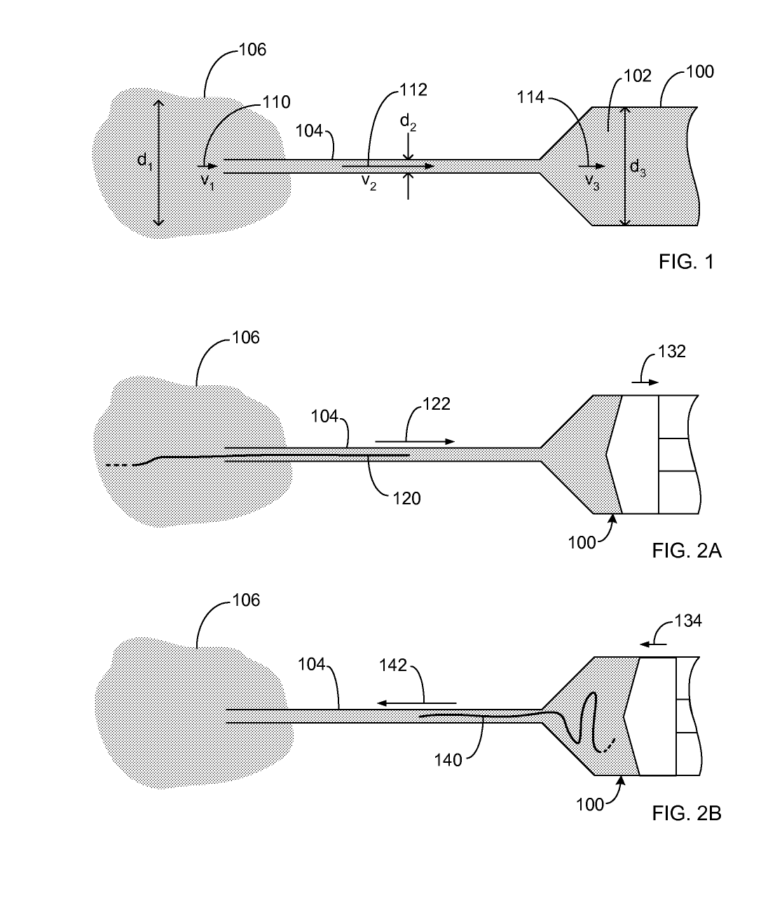

FIG. 1 shows an example situation where a volume of fluid 106 is in communication with another volume of fluid 102 via a conduit such as a needle 104. The volume 106 having a representative dimension d₁ can be inside a body (e.g., at or near suturing site) or in a container outside of the body. The volume 102 having a representative dimension d₃ can be within a suture passer device 100 having one or more features of the present disclosure.

In FIG. 1, the needle 104 is depicted as having an inner dimension d₂. Typically, such dimension is much less than those associated with the volumes 106 and 102. Thus, if fluid flow is induced from the outside (of the suture passer) volume 106 to the inner volume 102—via, for example, drawing motion of the suture passer 100—fluid flow speed inside the needle 104 will be substantially greater than speeds associated with the volumes 106 and 102. If the outer volume 106, inside of the needle 104, and inner volume have respective sectional areas A₁, A₂, and A₃, flow speed v₂ inside the needle 104 can be estimated as v₂=(A₁/A₂)v₁=(A₃/A₂)v₃ based on Venturi effect. Thus, relatively high flow speed v₂ (depicted as arrow 112) can be achieved with relatively slow drawing speed v₃ (e.g., drawing speed of a plunger) (depicted as arrow 114) due to a relatively high ratio A₃/A₂. In reverse, relatively slow expelling motion in the inner volume 102 can also result in relatively high flow speed of fluid being expelled through the needle 104.

If a suture is introduced in the fluid flow inside the needle, it can be carried by the fluid in an effective manner. In FIG. 2A, an end of a suture 120 is depicted as being carried (depicted by arrow 122) by the fluid inside the needle 104 as the fluid is being drawn into the suture passer 100. The speed of the suture 120 (depicted as arrow 122) may not be as great as the fluid flow speed; however, the suture's speed in the needle 104 will likely be greater than the speed associated with drawing motion (depicted as arrow 132) of the suture passer 100.

In FIG. 2B, an end of a suture 140 is depicted as being carried (depicted by arrow 142) by the fluid inside the needle 104 as the fluid is being expelled from the suture passer 100. The speed of the suture 140 (depicted as arrow 142) may not be as great as the fluid flow speed; however, the suture's speed in the needle 104 will likely be greater than the speed associated with expelling motion (depicted as arrow 134) of the suture passer 100.

As shown in FIGS. 2A and 2B, suture ends introduced into a conduit such as a needle allows the suture to be passed by being pulled into (FIG. 2A) or from (FIG. 2B) the suture passer. Such pulling of one end of the suture not only allows the suture to be passed to a desire location quickly, but also allows such passing to be achieved with less likelihood of tangling (of the remainder of suture being pulled).

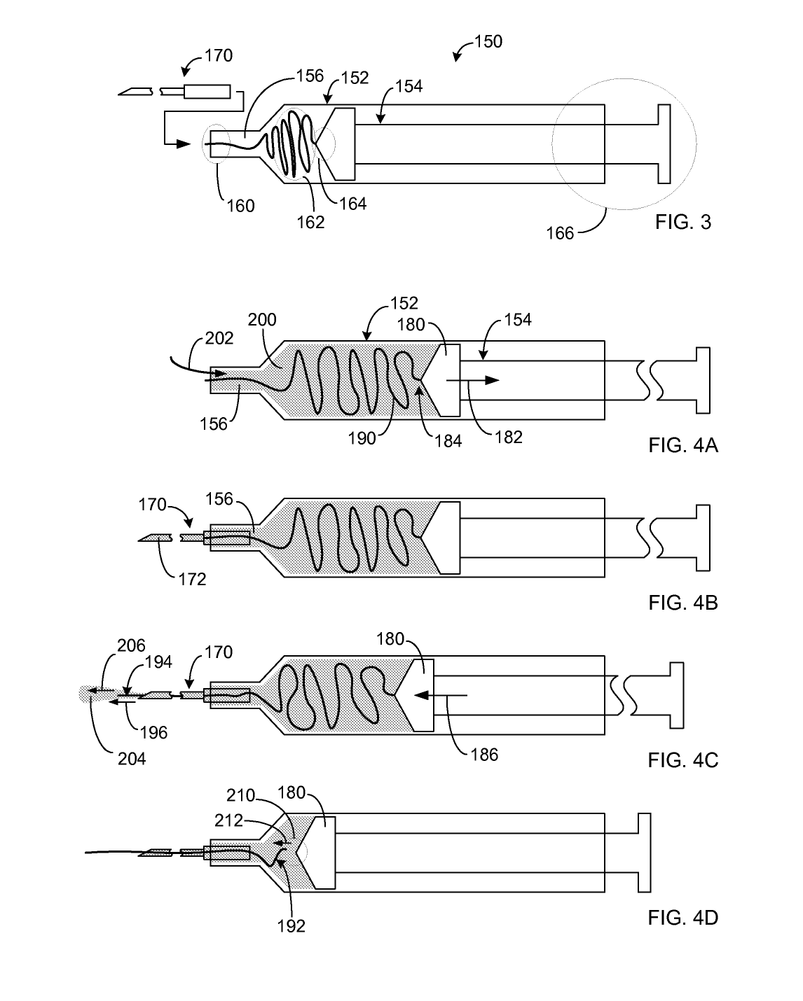

Described herein are various features that can enhance the use of suture passer devices. FIG. 3 shows that in certain embodiments, a suture passer 150 can include a barrel 152 and a corresponding plunger 154 that can facilitate drawing and expelling of fluid into and out of the suture passer 150. In certain embodiments, a suture can be positioned inside the suture passer 150 so that one end is secured to the plunger 154 in a releasable manner. In certain embodiments, the other end of the suture can be positioned relative to an end conduit 156 of the barrel 152 so as to facilitate positioning of that suture end into a suture passing conduit such as a needle 170.

As described herein, the suture passer 150 can include one or more features that can enhance positioning of the suture relative to the end conduit 156 (depicted as reference numeral 160), positioning of the suture within the suture passer 150 (depicted as reference numeral 162), releasably securing the suture end to the plunger 154 (depicted as reference numeral 164), ergonomical use of the suture passer 150 (depicted as reference numeral 166), or some combination thereof.

FIGS. 4A-4D show an example sequence of steps that can be taken when using a suture passer having a release mechanism (164 in FIG. 3). In certain embodiments, the suture passer can be packaged with suture 190 pre-loaded substantially inside a barrel 152. Such packaging can be achieved so as to provide a sterile suture passer during a surgical procedure.

In certain embodiments, the suture passer can be packaged dry, so that fluid 200 can be introduced into the suture passer for operation. Thus, FIG. 4A shows drawing in (depicted as arrow 202) of the fluid 200 from outside of the suture passer via a drawing motion (depicted as arrow 182) of a plunger 154. As shown, the fluid 200 is being drawn in through a relatively narrow conduit 156 at the end of the barrel 152; and thus, flow speed in the conduit 156 will likely be higher than at the larger-dimensioned portion of the barrel 152. Consequently, a portion of the suture 190 positioned in the conduit 156 (to, for example, facilitate insertion into a needle) will have a strong tendency to be pushed into the barrel 152 as fluid is being drawn in. Although not shown in FIGS. 4A-4C, one or more features can be provided to the conduit 156, to the needle, or any combination thereof, so as to inhibit such retraction of the suture end into the barrel when fluid is being drawn in. Examples of such features are described herein in greater detail.

In certain embodiments, the plunger 154 can include a tip 180 configured to provide a movable seal for the purpose drawing and expelling fluid into and from the interior of the barrel 152. Such a tip can be formed in a number of ways from materials based on silicone, rubber, plastic, etc.

In certain embodiments, one end of the suture 190 can be retained (depicted as reference numeral 184) in a releasable manner by one or more features formed or attached to the tip 180. Various examples of such releasable retaining of suture are described herein in greater detail. As shown in FIG. 4A and described herein, retaining the suture end at the plunger tip 180 can be beneficial as the plunger 154 is drawn away from the conduit 156. During such drawing motion of the plunger, relatively fast flowing fluid 200 entering the barrel 152 can result in bunching up of the suture 190. However, the suture end being moved away from the conduit 156 can mitigate or reduce the likelihood of the suture 190 becoming tangled.

In certain embodiments where the suture end is retained at or about the conduit 156, likelihood of suture tangling (when fluid is drawn in) can be further reduced, since both ends of the suture are secured and such securing locations are being separated. In the event that suture tangling occurs, such securing of both ends can also be beneficial, since the tangling will likely result in a detectable resistance as the plunger 154 is being drawn.

In FIG. 4B, fluid has been drawn into the suture passer, and the needle 170 is shown to be coupled to the conduit 156 portion of the barrel 152. Further, the suture end is shown to be inserted into the coupling end of the needle 170. In certain embodiments, the needle 170 can define an interior pathway 172 dimensioned to allow passage of the suture being carried by the fluid. The tip of the needle can be configured to allow relatively easy penetration through tissue or any material being sutured. Such penetration can be achieved with or without a suture extending out of the needle's tip.

In FIG. 4B, the needle's interior pathway 172 is depicted as being occupied by the fluid (shaded pattern). Such filling of the needle can be achieved, for example, by expelling a small amount of the fluid from the barrel. Such expelling motion can also position the suture end further into the needle from the needle's coupling end.

In certain situations, the needle 170 can be introduced to a suturing site inside a body, either through a cannula or by direct penetration through tissue. Such introduction of the needle 170 to the suturing site can be performed with the example suture passer configuration depicted in FIG. 4B.

In FIG. 4C, the plunger tip 180 is depicted as being moved (arrow 186) so as to expel the fluid from within the barrel to a location outside of the needle 170. More particularly, expelled fluid 204 leaving (arrow 206) the needle tip is shown to move (arrow 196) the leading end 194 of the suture out of the needle tip.

In certain situations, such expelling of the suture end from the needle tip can pass the suture end from first side of tissue to second side. Subsequent to such passing of the suture end, one or more additional expelling actions may be performed to position the suture in a variety of configurations. For example, the needle 170 can be retracted back to the first side of the tissue, and the suture end on the second side can remain there via suture-tissue friction and/or some other method (e.g., by a clamp). As the needle is retracted, the suture within the needle and the barrel can be pulled out relatively easily so as to facilitate retaining of the suture end on the second side. Once the needle tip is back on the first side, the needle can be passed through the tissue again with the suture folded over the end of the needle tip. Once the needle tip is on the second side, fluid and more suture can be expelled from the suture passer so as to form a suture loop. Such loop forming process can be repeated as desired or needed.

In FIG. 4D, the retained end 192 of the suture is depicted as being released (arrow 212) from the plunger tip 180. Various examples of such release (depicted as reference numeral 210) are described herein in greater detail. In certain embodiments, such release can be achieved with sufficient expelling capacity of the suture passer so as to allow passing of the released suture end to be expelled from the barrel.

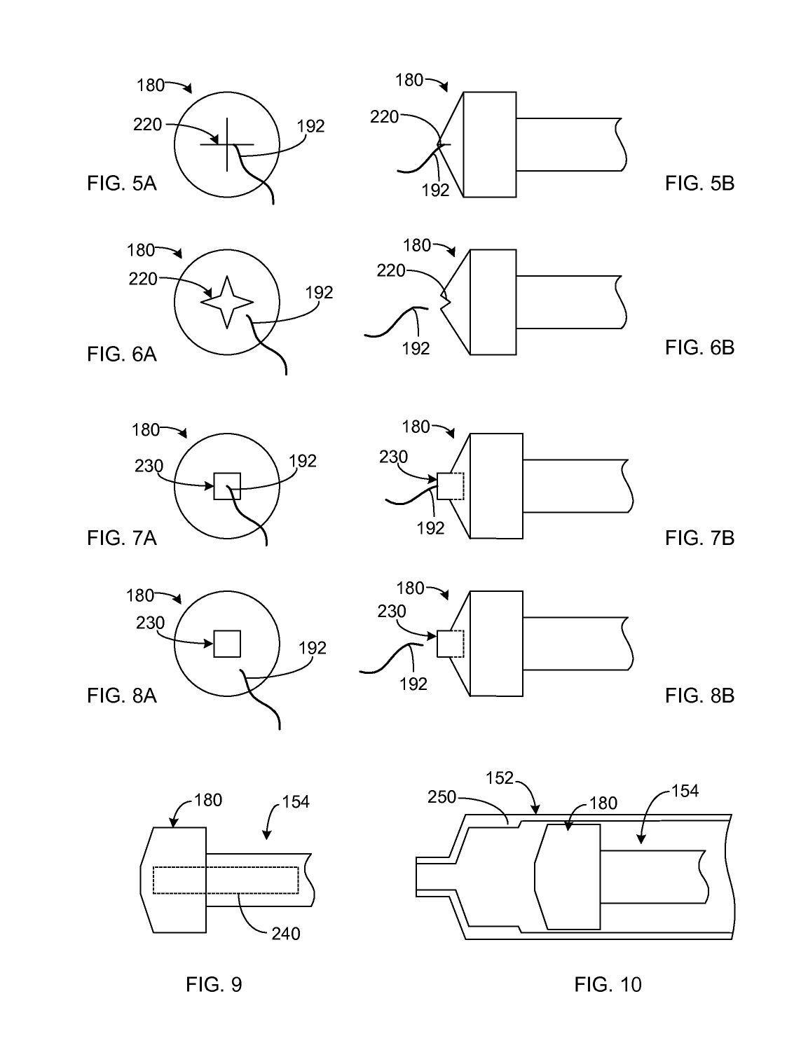

FIGS. 5-7 show that in certain embodiments, a mechanism that releasably retains one end of a suture can include one or more features that are integral part of a plunger tip, an additional structure that is coupled to the plunger tip, or some combination thereof. FIGS. 5A-6B show end and side views of a plunger tip 180 having one or more features 220 that are formed as part of the plunger tip so as to releasably retain one end of a suture 192. In FIGS. 5A and 5B, the one or more features 220 are in a retaining configuration that retains the suture end. In FIGS. 6A and 6B, the one or more features 220 are in a releasing configuration that releases the suture end. Examples of such one or more features are described herein in greater detail.

FIGS. 7A-8B show end and side views of a plunger tip 180 having a retaining member 230 positioned thereon so as to releasably retain one end of a suture 192. In FIGS. 7A and 7B, the retaining member 230 is in a retaining configuration that retains the suture end. In FIGS. 8A and 8B, the retaining member 230 is in a releasing configuration that releases the suture end. Examples of such a retaining member are described herein in greater detail.

FIGS. 9 and 10 show that in certain embodiments, a mechanism that releases the suture end can be actuated by one or more features associated with a plunger, by an interaction between a plunger and a barrel, or some combination thereof. FIG. 9 shows that in certain embodiments, a plunger tip 180 having suture retaining and releasing capability can have the suture release actuated by a release actuator component 240 that is part of the plunger 154. Examples of such an actuation configuration are described herein in greater detail

FIG. 10 shows that in certain embodiments, a plunger tip 180 having suture retaining and releasing capability can have the suture release actuated via an interaction of the plunger 154 with one or more features associated with the barrel 152. In certain embodiments, the plunger tip 180 can interact with a structure 250 defined by the barrel 152 so as to actuate the suture release. Examples of such an actuation configuration are described herein in greater detail.

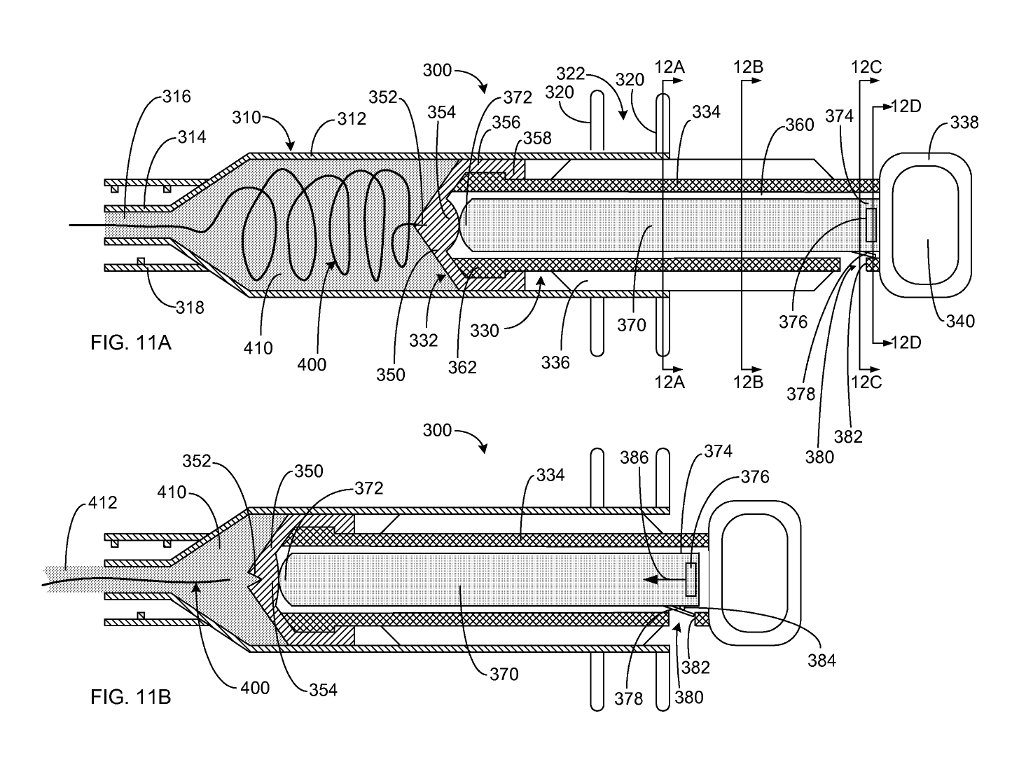

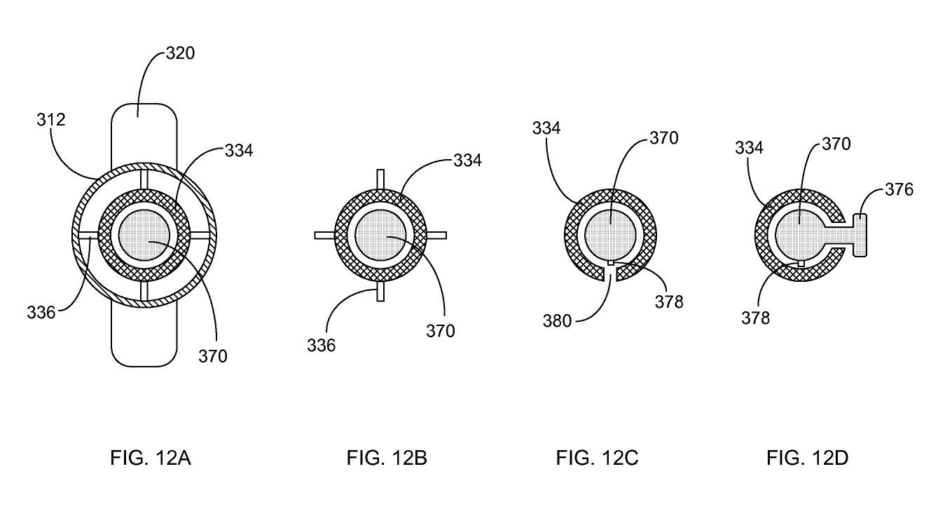

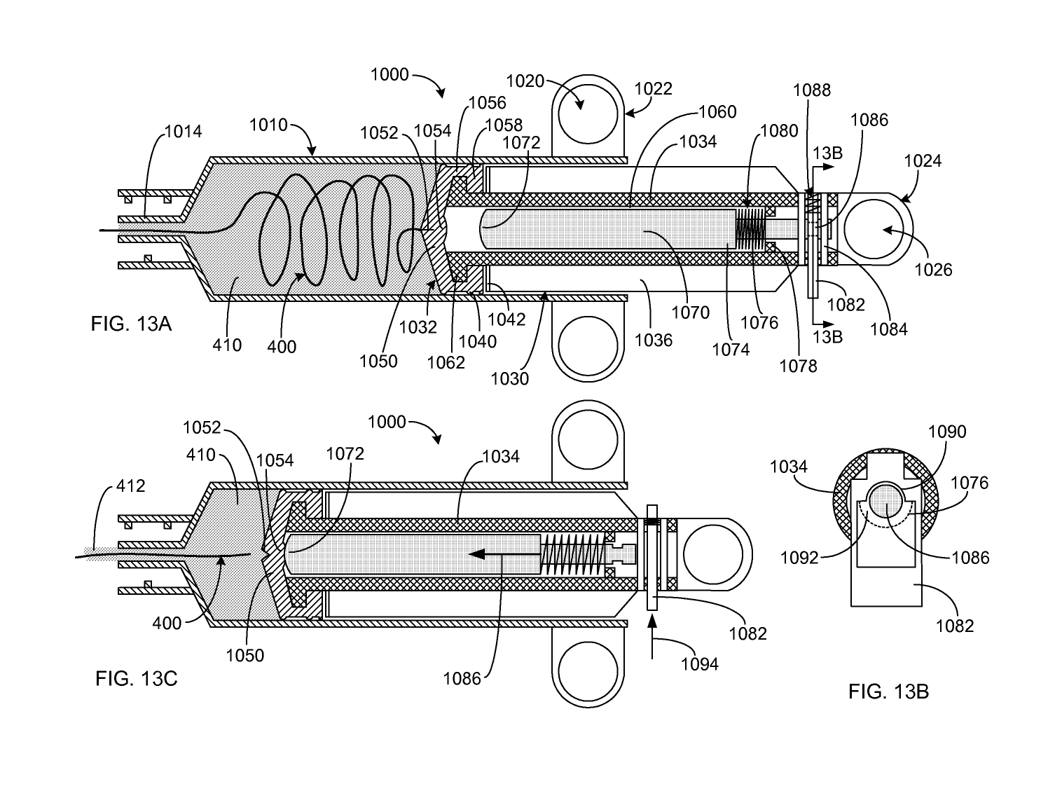

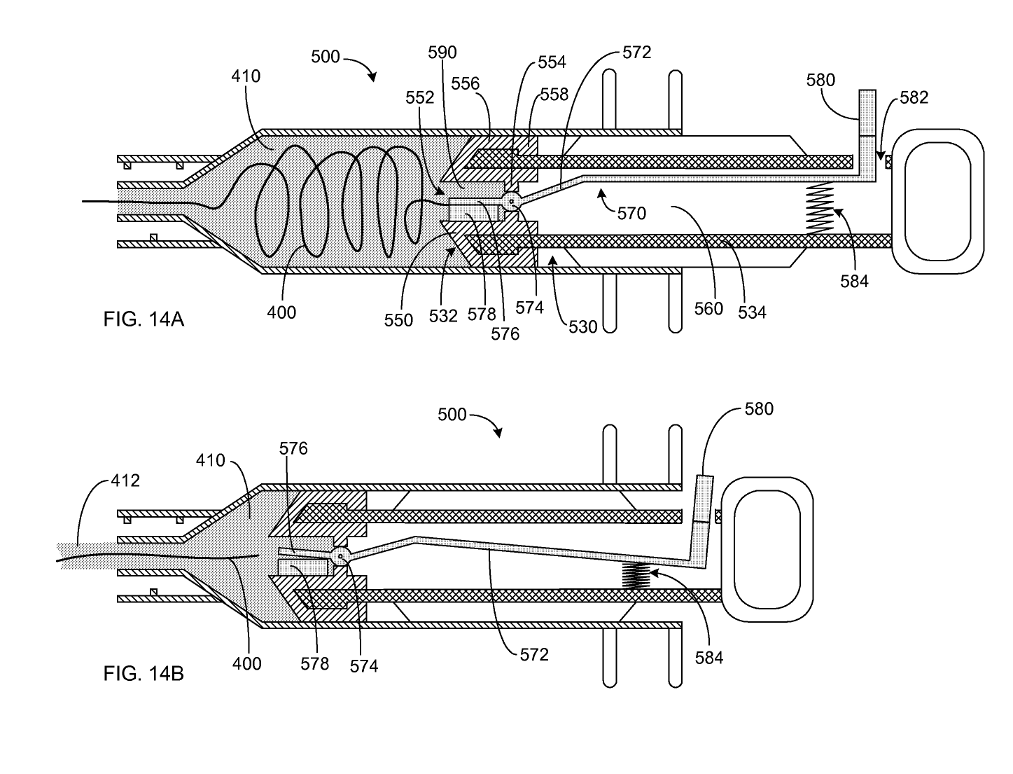

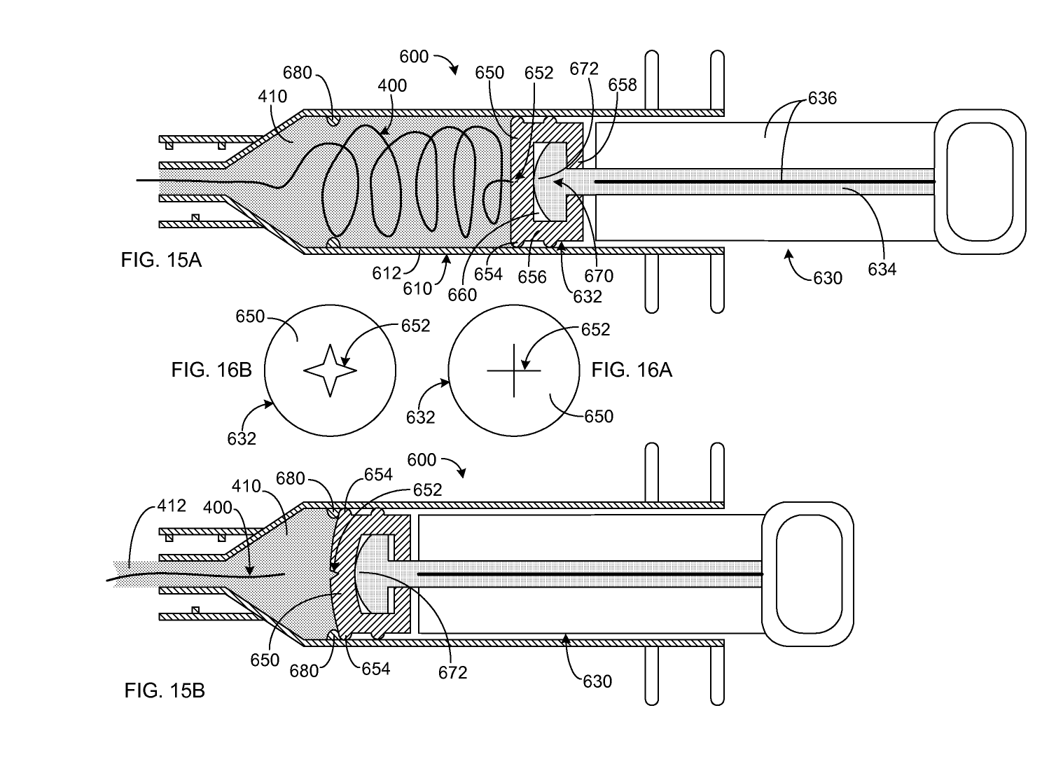

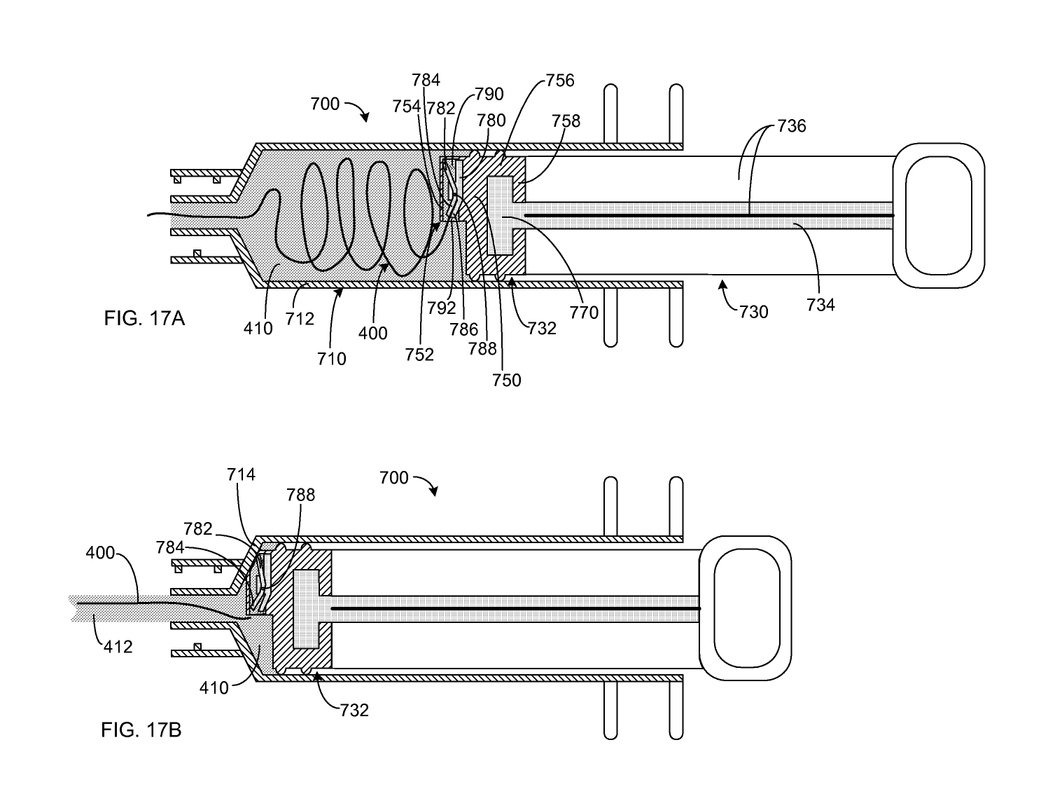

FIGS. 11-17 show non-limiting examples of suture releasing mechanism and actuation configurations. FIGS. 11 and 12 show an example suture passer 300 where a suture releasing mechanism can be part of a plunger tip, and where actuation of such a mechanism can be achieved by an actuator that is part of the plunger. FIG. 13 shows an example suture passer 1000 where a suture releasing mechanism can be part of a plunger tip, and where actuation of such a mechanism can be achieved by a spring loaded actuator that is part of the plunger. FIG. 14 shows an example suture passer 500 where a suture releasing mechanism can be provided to or about a plunger tip, and where actuation of such a mechanism can be achieved by an actuator that is part of the plunger. FIGS. 15 and 16 show an example suture passer 600 where a suture releasing mechanism can be part of a plunger tip, and where actuation of such a mechanism can be achieved by an interaction of the plunger tip with a feature formed on a barrel. FIG. 17 shows an example suture passer 700 where a suture releasing mechanism can be provided to or about a plunger tip, and where actuation of such a mechanism can be achieved by an interaction of the releasing mechanism with a feature associated with a barrel.

FIG. 11A shows a side sectional view of an example suture passer 300 where one end of a suture 400 is releasably retained by a suture holding mechanism 352. FIG. 11 B shows a side sectional view of the suture passer 300 where the previously retained suture end is released from the suture holding mechanism 352. Configurations of the suture passer 300 depicted in FIGS. 11A and 11B can represent, for example, operational steps described herein in reference to FIGS. 4B and 4D (without the needle).

Cross-sectional views indicated as 12A-12D are shown in FIGS. 12A-12D, respectively.

FIG. 11A shows that in certain embodiments, the suture passer 300 can include a barrel assembly 310 coupled to a plunger assembly 330 so as to define a chamber capable of being filled with fluid 410. For the purpose of description, it will be assumed that the fluid 410 has been drawn into the chamber, or already existed in the chamber. Also for the purpose of description, one end of the suture 400 is shown be releasably secured to the plunger 330 at one end; and the other end of the suture 400 is shown to be positioned in a flow conduit 316 defined by a tip 314 of the barrel 310. Such an end of the suture can be introduced into a needle (not shown) and passed through the needle as described herein in reference to FIGS. 4A-4D. In certain embodiments, such a needle can be coupled to the tip 314 via a securing mechanism 318 such as a Luer lock.

In certain embodiments, the barrel 310 include wall 312 that defines the chamber therein (e.g., a cylindrical chamber). The cylindrical portion of the barrel can be tapered to the tip portion 314 so as to define the reduced diameter conduit 316. The inner diameter of the wall 312 can be selected so as to allow receiving of the plunger 330, and allow piston-like motion of a plunger tip 332 within the barrel 310.

In certain embodiments, the barrel 310 can also include one or more ergonomic features such as finger support features 320 that can facilitate one-handed operation of moving the plunger 330 in and out of the barrel 310. The support features 320 define spaces 322 dimensioned to receive fingers (e.g., index and middle fingers). The example spaces 322 are depicted as being opened; however, it will be understood that similar finger receiving spaces can also be closed (e.g., by finger support features such as rings).

In certain embodiments, the plunger assembly 330 can include the tip 332 coupled to a hollow rod 334 that defines an axially extending bore 360. The rod can be dimensioned at the tip end so as to securely hold the tip 332. For example, the tip 332 can include a front portion 350, a side portion 356, and a rear portion 358 that define a recess dimensioned to receive an increased diameter portion 362 of the rod 334. More particularly, the rear portion 358 extending towards the axis of the rod 334 engages with the increased diameter portion 362 so as to inhibit the tip 332 from slipping off of the rod 334.

In certain embodiments, the plunger assembly 330 can include a plurality of wings 336 disposed on the outer surface of the hollow rod 334. For example, four such wings (336) can be dimensioned so as to maintain the generally coaxial alignment of the plunger 330 and the barrel 310. FIGS. 12A and 12B show additional views of the example wings 336 relative to the hollow rod 334 and the barrel wall 312.

As shown in FIG. 11A, the bore 360 of the rod 334 can be dimensioned to receive an actuator rod 370 having a tip end 372. The actuator rod 370 can move axially within the bore 360 so as to allow engagement of the tip end 372 with the front portion 350 of the plunger tip 332. Such engagement can include the tip end 372 pushing against the front portion 350 so as to deform the front portion 350 outward and open one or more features formed on the front portion 350. Such opening action and releasing of the suture are described in greater detail in reference to FIG. 11B.

In certain embodiments, the tip end 372 and/or the inner side of the front portion 350 can be shaped to facilitate the outward deformation upon engagement. For example, the inner side of the front portion 350 of the plunger tip 332 can include a shaped bump 354 to promote deformation. In certain embodiments, such a bump (354) can be dimensioned and positioned such that its peak is located behind the one or more suture-retaining features (352). For example, if such features are generally centered about the longitudinal axis of the plunger 330, the peak of the bump 354 can be disposed at or near the same axis.

In another example, the tip end 372 of the actuator 370 can be shaped to promote deformation as it engages the front portion 350 of the plunger tip 332. In the example shown, the actuator tip 372 is shown to be rounded symmetrically such that the rounded tip generally engages the center of the bump 354. Such an engagement configuration can promote deformation of the front portion 350 by providing room for deformation about the engagement location.

As shown in FIG. 11A, the hollow rod 334 can be attached to a plunger end, and such an end can include an ergonomic feature 338 such as a ring structure. The example ring 338 defines an opening that can be dimensioned to receive a finger (e.g., a thumb) to allow one-handed operation of the suture passer device 300.

The finger ring 338 can be attached to the hollow rod 334 so as to provide a generally rigid mechanical coupling between the finger ring 338 and the tip end 362 of the hollow rod 334. Thus, pulling and pushing motions of the finger ring 338 effectuates drawing in of fluid into the chamber and expelling of fluid from the chamber, respectively.

In certain embodiments, the actuator rod 370 is not attached to the finger ring 338 so as to allow the rod 370 to be substantially free to move axially within the bore 360 of the hollow rod 334. Thus, the rod 370 can be pushed axially relative to the hollow rod 334 (and thus the finger ring 338 and the plunger tip 332) to deform the front portion of the plunger tip 332.

Such pushing of the actuator rod 370 can be achieved in a number of ways. For example, a pushing tab 376 can be disposed at or near the rod end 374 (proximate to the finger ring 338). Thus, pushing of the tab along a direction having an axial component can result in the actuator rod 370 engaging and deforming the front portion 350 of the plunger tip 332. An additional view of the tab 376 and its relation to the actuator rod 370 is depicted in FIG. 12D.

In certain embodiments, the plunger assembly 330 can include a functionality where the actuated rod 370 can remain in its pushed configuration. For example, a spring (384) loaded arm 378 can be disposed relative to an opening 380 defined by the hollow rod 334. One end of the arm 378 can be secured to the actuator rod 370, and spring loading can be such that the other end of the arm 378 wants to move away from the rod 370. When the arm 378 is constrained in within the bore 360 of the hollow rod 334, the actuator rod 370 is generally free to move within the bore 360. However, when the loaded arm 378 encounters the opening 380 (e.g., when the actuator rod 370 is pushed), the unsecured end of the arm 378 moves away from the rod 370 and catches on one of the edges of the opening 380 thereby partially inhibiting movement of the actuator rod 370 relative to the hollow rod 334. FIGS. 12C and 12D show additional views of the loaded arm 378 and its relation to the hollow rod 334 and the opening 380.

In the example shown in FIG. 11B, the actuator rod 370 is depicted as having been pushed (arrow 386) so as to deform the front portion 350 of the plunger tip 332. Accordingly, the unsecured end of the loaded arm 378 is shown to have caught the rear edge of the opening 380 so as to maintain the pushed configuration and inhibit the actuator rod 370 from moving back relative to the hollow rod 334.

It will be understood that other configurations of movability of actuator rod 370 may be desired. For example, it may be desirable to have the plunger tip 332 be deformed only as long as the pressure is applied to the tab 376. In such a situation, the loaded arm 378 or similar device can be omitted from the suture passer device 300. In another example, it may be desirable to have such a loaded arm be retractable so as to allow restoring of the actuator rod 370 back to its “un-pushed” configuration. In such a situation, the loaded arm 378 or similar device may be provided with a retraction device that, for example, pushes the loaded arm 378 back towards the rod 370.

As shown in FIG. 11B, deformation of the plunger tip 332 can be achieved by a pushing force applied by the actuator rod 370. The tip deformation is shown to result in the suture retaining feature 352 to open up and release the end of the suture 400. The released suture 400 is depicted as being carried by a flow 412 of fluid through the needle (not shown).

In certain situations, such releasing of the suture 400 from the suture passer 300 can be desirable. Some non-limiting examples of such situations are described herein in greater detail.

For the example suture passer 300 shown in FIGS. 11 and 12, the releasing action is substantially within the plunger assembly 330. Thus, the suture 400 can be released from the release mechanism 352 regardless of the plunger's position relative to the barrel. In certain situations, and as shown in FIG. 11B, the release of suture 400 is performed when the plunger 332 is near or approaching the tip 314 of the barrel 310. In some of such situations, it may be desirable to release the suture 400 while there is sufficient expellable fluid 410 left in the chamber, so as to allow the remaining expelling fluid to carry the suture end out of the chamber and the needle.

FIGS. 13A -13C show another example suture passer 1000 where a suture releasing mechanism can be part of a plunger tip 1032, and where actuation of such a mechanism can be achieved by a spring loaded actuator that is part of the plunger 1030.

As shown, a barrel 1010 and associated parts such as a reduced diameter conduit 1014 can be similar to those described herein in reference to FIGS. 11A and 11B. Similarly, the plunger tip 1032 and its coupling to a hollow rod 1034 can also be similar to those described in reference to FIGS. 11A and 11B.

FIG. 13A shows an example configuration of the suture passer 1000 where one end of the suture 400 is retained by a retaining mechanism 1052 formed on the plunger tip 1032. FIG. 13B shows the suture 400 being released when the retaining mechanism 1052 opens up due to deformation of the plunger tip 1032 induced by the actuator rod 1070.

FIG. 13A shows that in certain embodiments, the actuator rod 1070 can be spring loaded (by a spring 1080) so as to be in a “cocked” and retracted configuration when the suture retaining mechanism is closed. Such cocked and retracted configuration can be maintained by a trigger mechanism, an example of which is shown in FIGS. 13A-13C.

As shown in FIG. 13A, the actuator rod 1070 can include a tip engaging end 1072 and a rear portion disposed near the finger ring 1024. The rear portion of the rod 1070 can include a reduced diameter portion 1076. As shown, a coil spring 1080, having an inner diameter selected to fit over the reduced diameter portion 1076, can be provided. The spring's inner diameter being less than the diameter of the normal-diameter portion 1074 allows one end of the spring 1080 to be retained by the step formed between the two different diameter portions. The spring's outer diameter can be selected to fit within the bore 1060 of the hollow rod 1034.

As shown in FIG. 13A, the other end of the spring 1080 can be retained by a retaining feature 1078 that also defines a reduced diameter aperture for passage of the reduced diameter portion 1076. The reduced diameter portion 1076 is shown to extend through the aperture and engage a triggering mechanism.

In certain embodiments as shown in FIGS. 13A and 13B, the triggering mechanism can include a trigger 1082 that defines a variable-width slot (depicted as 1090 and 1092) and coupled to the hollow rod 1034 so as to be movable laterally (depicted as arrow 1094 in FIG. 13C). When the actuator rod 1070 is in its cocked and retracted position (FIG. 13A), the trigger 1082 is in a position so that the smaller dimensioned portion of the slot 1090 engages a groove 1086 formed on the reduced diameter portion 1076 of the rod 1070 to thereby inhibit forward movement of the rod 1070. Pushing the trigger 1082 along the direction 1094 (FIG. 13C) results in the reduced diameter portion 1076 of the rod 1070 being able to move through the larger dimensioned portion of the slot 1092 to thereby allow forward movement (arrow 1086 in FIG. 13C) of the rod 1070 from de-compressing of the spring 1080.

In certain embodiments, the trigger 1082 can be coupled to a trigger spring 1088 so as to reduce likelihood of accidental release and/or for improved tactile control of the trigger 1082. In certain embodiments, one or more other features (such as trigger surface features) that promote desired resistance and tactile response of the triggering action can be implemented.

As shown in FIG. 13C, the spring induced forward motion of the actuator rod 1070 engages and deforms the plunger tip 1032 so as to release the suture 400 in a manner similar to that described in reference to FIG. 11B. In the example shown in FIG. 13C, the spring 1080 having been restored closer to its rest configuration can inhibit the spring 1080 from being compressed again due to the restoring force of the plunger tip 1032. In certain embodiments, the spring strength and deformation characteristics (e.g., material and/or dimensions) of the plunger tip 1032 can be selected so as to achieve a desired actuation configuration.

As shown in FIGS. 13A and 13C, the example suture passer 1000 is depicted as having ergonomic features such as finger rings 1022 and 1024. Such rings can provide similar functionalities as the example finger support features (320) describe herein in reference to FIGS. 11A and 11B. It will be understood that various examples of the suture passer as described herein can have either of the ergonomic features, some part or combination thereof, other types of ergonomic features, or none at all.

FIG. 14 shows an example suture passer 500 where a suture releasing mechanism 552 can be provided to or about a plunger tip 532, and where actuation of such a mechanism can be achieved by an actuator that is part of the plunger 530.

As shown, a barrel and associated parts can be similar to those described herein in reference to FIGS. 11A and 11B. Similarly, passage of the suture 400 before and after release from the release mechanism 552 can be similar to that described herein in reference to FIGS. 11A and 11B.

In the example configuration of the suture passer 500, the plunger 530 can include the tip 532 dimensioned to couple to a hollow rod 534 and to receive the release mechanism 552. The plunger tip 532 can be dimensioned to have front (550), side (556), and rear (558) portions so as to be retained by the tip portion of the hollow rod 534.

As shown, the plunger tip 532 can also define a recess 590 that faces the fluid chamber and dimensioned to receive the release mechanism 552. The release mechanism 552 can be a jaw structure having first and second jaw members 576 and 578, and the jaw can be biased in a closed position and retain the suture 400 by a pivotable coupling arm 572 and a spring 584.

In certain embodiments, pivoting of the coupling arm 572 and the first jaw member 576 can be facilitated by a pivot assembly 574. In certain embodiments, area about the pivot assembly 574 can be sealed from fluid leakage by a sealing member 554.

As shown, an actuator 570 that includes the coupling arm 572 can reside in a space 560 defined by the hollow rod 534. The coupling arm 527 can be extended towards the rear portion of the plunger 530 and be coupled to the spring 584.

In the example configuration shown, the spring 584 positioned between the coupling arm 527 and the wall of the hollow rod 534 biases the coupling arm 527 so as to keep the jaw members 576 and 578 in a closed position. The coupling arm 527 can be pressed laterally by, for example, a tab 580 that extends through the hollow rod's wall so as to facilitate a convenient pressing action by the user.

FIG. 14B shows the result of such a pressing action. The coupling arm 572 being pressed near the rear portion of the plunger 530 results in pivoting of the first jaw member 576 to thereby open the jaw. The opened jaw results in the suture 400 being released.

FIGS. 15 and 16 show an example suture passer 600 where a suture releasing mechanism 652 can be part of a plunger tip 632, and where actuation of such a mechanism can be achieved by an interaction of the plunger tip 632 with a feature 680 formed on a barrel 610.

In FIG. 15A, the barrel 610 having a wall 612 and a reduced diameter conduit tip can be similar to the example barrel described herein in reference to FIGS. 11A and 11B. In certain embodiments, the barrel wall 612 can define a feature 680 that can cooperate with the plunger tip 632 so as to actuate the release of the suture 400. For example, the actuating feature 680 can be a bump that protrudes inwardly from the wall 612 so as to engage the edge portion (654) of the plunger tip 632 and deform the tip's front portion 650 (depicted in FIG. 15B). Such a deformation can result in the release mechanism 652 to open up (FIG. 16B) from its relaxed closed configuration (FIG. 16A). In certain embodiments, the bump 680 and the barrel wall 612 can be formed as a single piece by a process such as injection molding.

In certain embodiments, the plunger tip 632 can be a single piece that includes the deformable front portion 650, a side portion 656, and a rear portion 658 that define a space 660 for receiving and retaining a tip portion 670 of the plunger 630. For the example suture passer 600 shown in FIGS. 15A and 15B, the plunger assembly 630 can be considerably simpler due to its lack of internal actuating mechanism. Thus, the plunger 630 can include a rod member 634 that interconnects the tip portion 670 to the rear portion. As shown, the plunger 630 can also have a plurality of wings to generally keep the plunger centered relative to the barrel 610.

As shown, the tip portion 670 of the rod member 634 can include a rounded engagement tip 672 to promote the outward deformation of the front portion 650 of the plunger tip 632. In certain embodiments, the front portion 650 can also include a rounded bump facing the engagement tip 672 to further promote the deformation.

As shown in FIGS. 15A and 15B, the plunger tip 632 can define one or more sealing members (e.g., ring-like structure(s) formed on the side of the tip 632) that inhibit passage of fluids past the plunger tip 632. In certain embodiments, one of such sealing rings can interact with the actuating feature 680 to induce the suture-releasing deformation.

For example, the front ring-line feature 654 is depicted as engaging the actuating feature 680 in FIG. 15B. Such engagement will likely be felt by the user, at which time the plunger 630 can be pushed further to result in the suture-releasing deformation.

FIG. 17 shows an example suture passer 700 where a suture releasing mechanism 752 can be provided to or about a plunger tip 732, and where actuation of such a mechanism can be achieved by an interaction of the releasing mechanism 752 with a feature associated with a barrel 710.

In FIG. 17A, the barrel 710 having a wall 712 and a reduced diameter conduit tip can be similar to the example barrel described herein in reference to FIGS. 11A and 11B. In certain embodiments, a plunger 730 having the tip 732 can be generally similar to the plunger 630 (FIGS. 15A and 15B) with respect to the rod member 734 and wings 736.

In certain embodiments, coupling of the rod member 734 to the plunger tip 732 can be simpler than that associated with the plunger 630 of FIGS. 15A and 15B. For example, because there is no need to deform the front portion 750 of the plunger tip 732, the tip end 770 of the rod member 734 can be dimensioned to be substantially similar to a space defined by the front (750), side (756), and rear (758) portions of the plunger tip 732.

As shown, a suture release mechanism 752 can be disposed at or about the front portion 750 of the plunger tip 732. In certain embodiments, a portion of the release mechanism 752 can be embedded in the front portion 752. Preferably, such embedded portion does not puncture or degrade the fluid-sealing properties of the front portion 750.

In the example shown in FIG. 17A, the release mechanism 752 can include first and second jaw members 784 and 786 pivotably coupled via a pivot mechanism 788. The first jaw member 784 is depicted as being interconnected to a movable arm 782, and the second jaw member 786 is depicted as being interconnected to a base member 780 that is attached to the front portion 750 (e.g., by being embedded).

In FIG. 17A, a spring 790 that biases the movable arm 782 and the base member 780 apart results in the first and second jaw members 784 and 784 being in a closed position so as to retain the suture therein. In FIG. 17B, the movable arm 782 engaging the tapered wall 714 of the barrel 710 can depress the arm 782 so as to pivot the first jaw member 784 away from the second jaw member 786, thereby releasing the suture 400.

In certain embodiments, the release mechanism 752 can be partially enclosed by a deformable enclosure 754. Such an enclosure (754) can be formed as a part of the plunger tip 732 (e.g., by injection molding process). The example enclosure 754 can be dimensioned to allow passage of the suture 400 when released, and to reduce the likelihood of suture 400 being tangled with the release mechanism 752.

In the non-limiting examples of the suture passers shown in FIGS. 11-17, one end of the suture 400 is shown to be secured and released by various examples of the release mechanism. The remaining portion of the suture 400 is depicted as being generally unconstrained in the chamber.

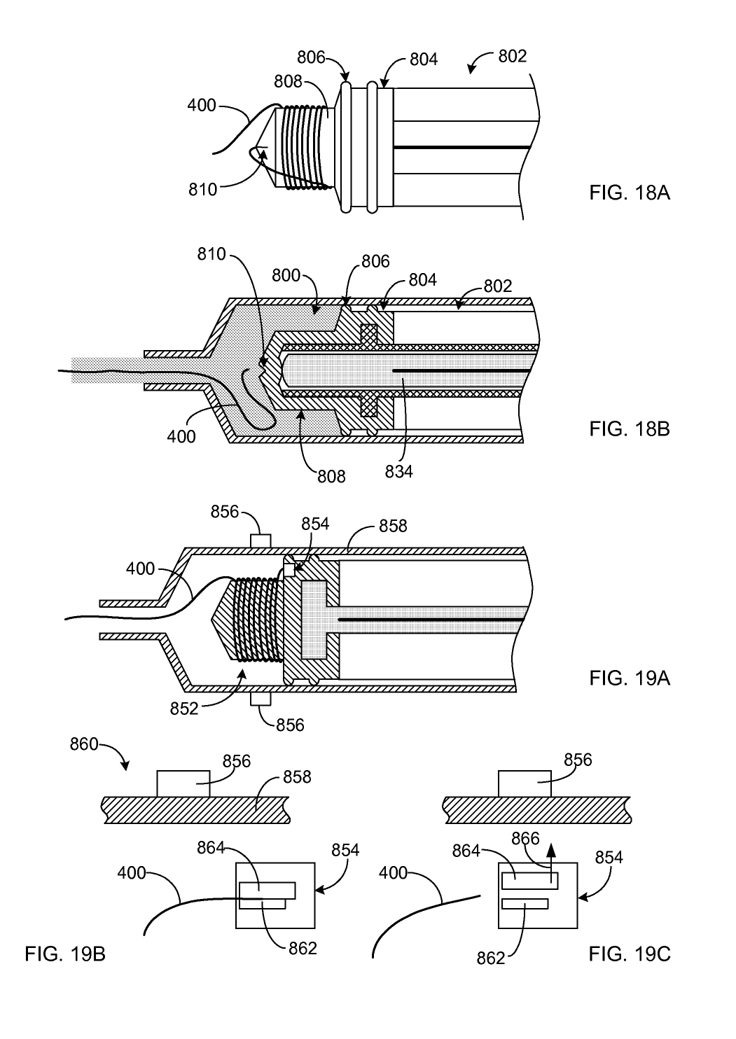

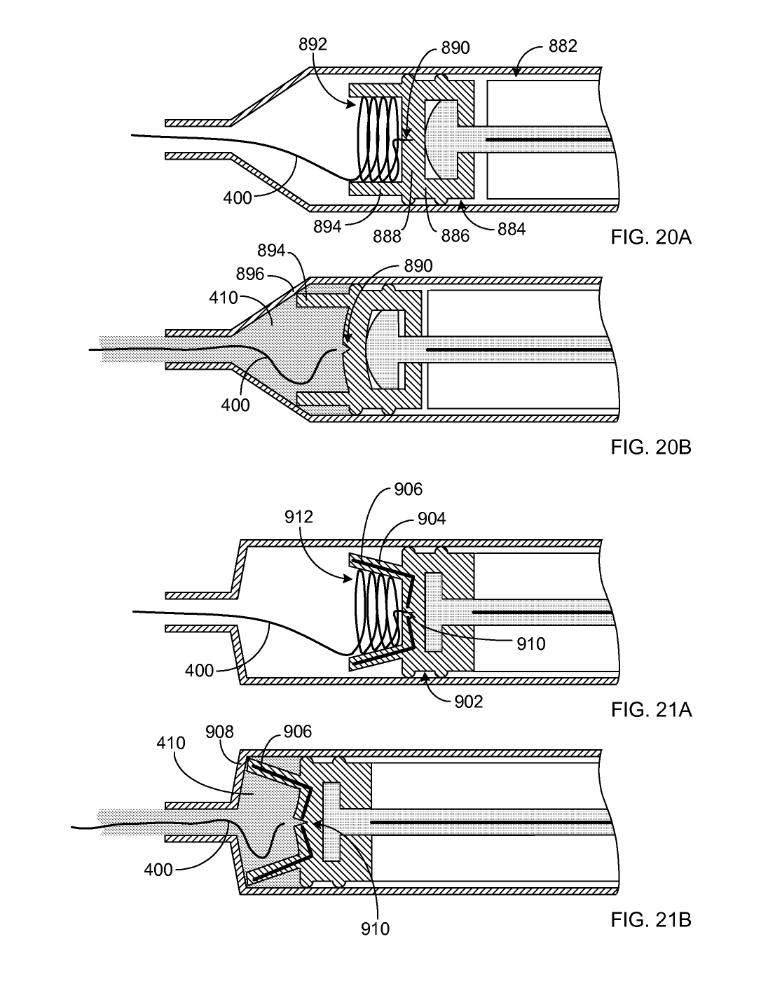

In certain embodiments, the suture can be arranged in the chamber via some feature associated with the suture passer device. Examples of such suture arrangement are shown in FIGS. 18 - 21. In FIGS. 18 and 19, examples of suture passers where the suture can be wound on a structure are shown. In FIGS. 20 and 21, examples of suture passers where the suture can be wound in a recess are shown.

In certain embodiments as shown in FIGS. 18A and 18B, a plunger 802 can include a tip 804 having a normal-diameter portion 806 (to provide, for example, piston-motion and fluid sealing functionalities) and a reduced diameter portion 808. In FIG. 18A, the suture 400 is depicted as being wound on the reduced diameter portion 808, and one end of the suture being retained by a release mechanism 810. In FIG. 18B, the suture 400 has been unwound, and its end is depicted as being released by the mechanism 810. In certain embodiments, such suture releasing mechanism can be actuated similarly to one or more examples disclosed herein. For example, the release and actuator mechanisms shown in FIG. 18B is similar to that described in reference to FIGS. 11 and 13.

In the example configuration shown in FIG. 18, the suture end securing and releasing location is forward of the winding location (on the reduced diameter portion 808). As such, there may arise a situation where the suture 400 may be wound on top of its end portion. For example, a winding (such as the example shown in FIG. 18A) that unwinds from the front can result in the suture 400 being wound on top of its end portion. In certain situations, it may be desirable to not have such a configuration.

For example, suppose that it is desirable to have the suture unwind from the front, and yet not have the suture be wound on top of itself. FIGS. 19A shows an example where such a configuration can be achieved in the context of the example reduced diameter portion (808 in FIGS. 18A and 18B).

In FIG. 19A, the suture 400 is shown to be wound about a reduced diameter portion 852, so as to allow unwinding from the front. The end of the suture 400 is depicted as being retained by a release mechanism 854 disposed to the rear of the reduced diameter portion 852, such that the suture winding is not on top of the suture end.

In certain embodiments, release of the suture 400 can be achieved by a mechanism that does not rely on deformation of the plunger tip. For example, suture release can be achieved by an interaction of the release mechanism 854 with one or more components 856 disposed on the barrel wall 858.

FIGS. 19B and 19C show enlarged views of the example interaction between the components 854 and 856. For example, the barrel component 856 can be a magnet that attracts and moves a first jaw member 864 when the release mechanism 854 is in its proximity. In FIG. 19B, the release mechanism 854 is sufficiently far enough from the magnet 856 so that the first and second jaw members 864 and 862 remain closed (e.g., by some biasing force such as a spring). In FIG. 19C, the release mechanism 854 is shown to be in close proximity to the magnet 856 (e.g., by the plunger tip passing by the magnet), such that the first jaw member 864 moves towards the magnet (arrow 866) to thereby release the suture 400.

In certain embodiments as shown in FIGS. 20A and 20B, a plunger 882 can include a tip 884 having a normal-diameter portion 806 (to provide, for example, piston-motion and fluid sealing functionalities). A front portion 888 of the plunger tip 884 and a forward extending wall 894 are shown to define a front-facing recess 892. In FIG. 20A, the suture 400 is depicted as being wound inside the recess 892, and one end of the suture being retained by a release mechanism 890 within the recess 892. In FIG. 18B, the suture 400 has been unwound, and its end is depicted as being released by the mechanism 890. In certain embodiments, such suture releasing mechanism can be actuated in part similar to that described in reference to FIGS. 15A and 15B. As shown in FIG. 18B, forward movement of the plunger tip 884 can be inhibited by the engagement of the wall 894 with the tapered portion 896 of the barrel, thereby allowing the plunger 882 to be pushed further and deform the front portion 888 of the tip 884.

FIGS. 21A and 21B show an example variation to the suture holder shown in FIGS. 20A and 20B. In certain embodiments, a front-facing recess 912 can be formed in a manner similar to that shown in FIG. 20A, and the suture 400 can be wound and retained therein.

In certain embodiments, the forward extending wall 904 can include one or more actuating members 906. For example, two “L” shaped members 906 are shown to be embedded in the wall 904 and the area leading up to the suture releasing mechanism 910. As shown in FIG. 21B, engagement of the front end of the L-shaped members 906 with the front portion 908 of the barrel can result in the plunger-pushing force being transferred to the other end of the L-shaped members 906 and opening up the release mechanism 910. In certain embodiments, the L-shaped members 906 can be formed from an appropriate material so as to be relatively rigid when compared to the deformation property of the plunger tip 902.

In certain situations, it may be desirable to know how much forward plunger travel is left so as to estimate the amount of fluid and possibly the suture remaining in the chamber. Generally, the user's thumb-to-fingers separation can provide such an estimate. There may be situations, however, where a tactile indication of the plunger's location relative to the barrel can be useful. For example, it may be desirable to know how much fluid remains in the chamber so as to allow release of the suture in a timely manner.

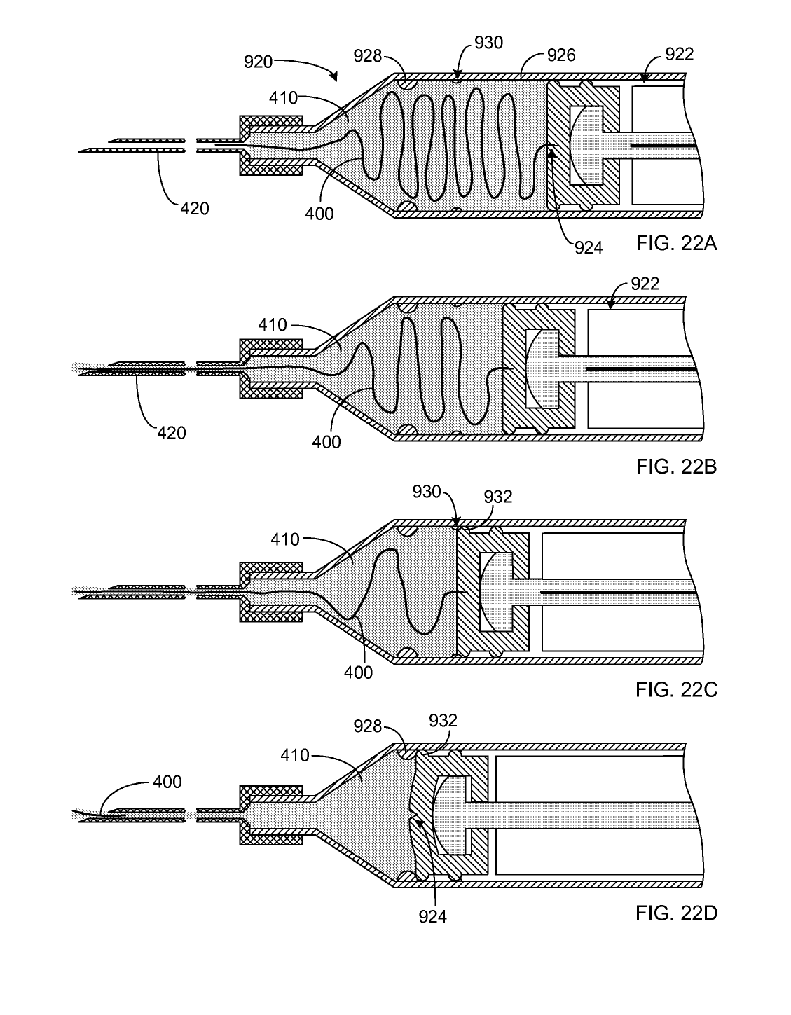

FIGS. 22A-22D show an example sequence of a suture passer 920 having a plunger position indicator 930. In certain embodiments, the position indicator 930 can be an appropriately sized bump formed on the inner surface of the barrel's wall 926. The position indicator 930 can be one or more bump structures or a ring-like structure distributed circumferentially around the wall 926.

In FIG. 22A, a plunger 922 is depicted as being in a relatively rearward position such that the chamber is filled with a relatively large amount of fluid 410. Similarly, a relatively lengthy suture 400 is positioned in the chamber, with one end retained by a release mechanism 924. The release mechanism 924 and the actuating mechanism can be similar to one or more mechanisms as described herein.

In FIG. 22A, the free end of the suture 400 is depicted as being introduced into the needle 420 so as to be ready to be expelled along with the fluid 410. In FIG. 22B, the plunger 922 is depicted as having been moved forward so as to result in some of the fluid 410 and the suture 400 being expelled.

In FIG. 22C, the plunger 922 has been moved forward such that a front edge portion 932 of the plunger's tip engages the position indicator 930. Such an engagement can provide a resistance that indicates to the user the relative location of the plunger 922.

In certain embodiments, the position indicator 930 can be dimensioned to provide sufficient resistance for the tactile input for the user, but not sufficient to inhibit further plunger movement or actuation of the suture release mechanism. Thus, in FIG. 22D, the plunger 922 is shown to have moved past the position indicator 930 so as to reach a desired release location. In the example shown, the example release mechanism is similar to that described in reference to FIGS. 15A and 15B. Accordingly, the front edge portion 932 is depicted as engaging actuating features 928 so as to release the suture 400.

In certain embodiments, the position indicator 930 can be located in the barrel so as to allow expelling of sufficient amount of fluid between the configurations shown in FIGS. 22C and 22D. Such “last” expelling motion can carry the released suture out of the barrel and the needle 420, as shown in FIG. 22D.

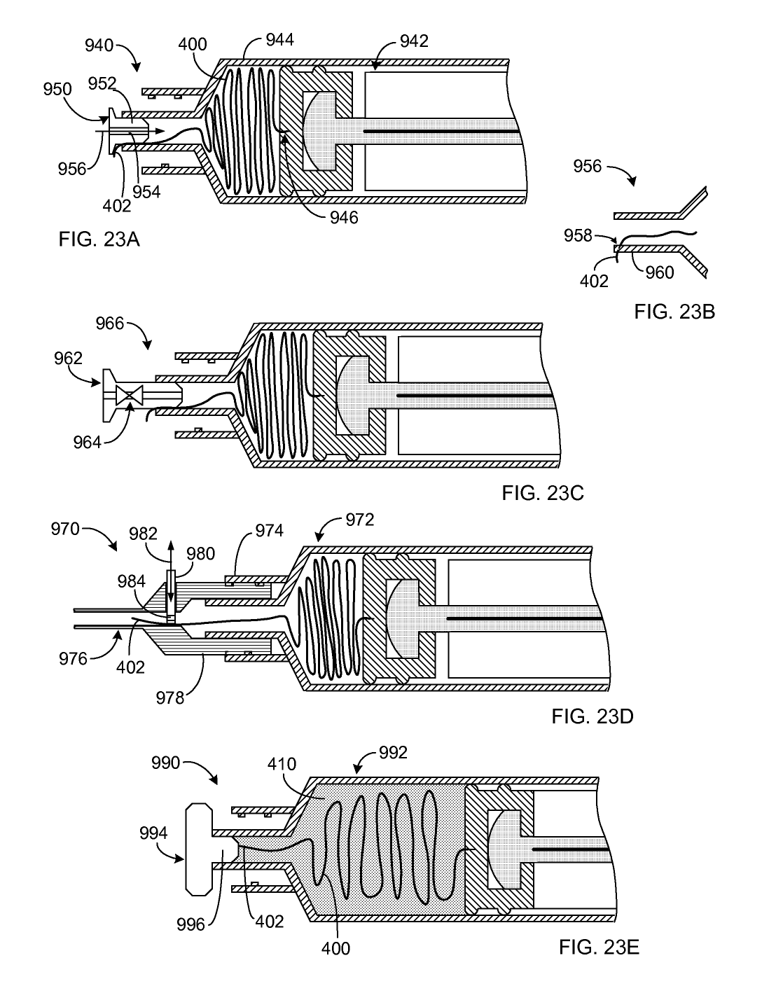

In certain embodiments, a suture passer device having one or more features of the present disclosure can be packaged with a suture 400 pre-loaded into the chamber. Preferably, such a packaged suture passer device is substantially sterile when removed from the package for use. In certain embodiments as shown in FIGS. 23A-23E, such suture-loaded passer device can be packed with or without a needle, and with or without fluid.

FIG. 23A shows a suture passer device 940 having a preloaded suture 400 and packaged dry. In certain embodiments, the plunger 942 can be in a relatively forward position, such that the suture 400 is relatively compacted near the front of the barrel 944. One end of the suture 400 is shown to be secured to a release mechanism 946 (e.g., during assembly prior to packaging). The free end 402 of the suture 400 is shown to be positioned outside of the reduced diameter conduit so as to facilitate its introduction into a needle (not shown).

In use, the suture-loaded passer 940 can be removed and filled with fluid from a container (e.g., outside the body) with or without a needle. In FIG. 23A, it is assumed that such a filling is performed with the needle. During the filling process, it is desirable to retain the free end 402 of the suture 400 outside of the conduit. Thus, the suture passer 940 can be accompanied with a plug 950 that fits into the conduit so as to wedge the suture end 402 between it and the conduit wall. In certain embodiments, the plug 950 can define an aperture 954 that allows passage of fluid being drawn in (depicted as arrow 956) while retaining the suture end 402. Once fluid has been drawn in, the plug 950 can be removed, and the suture end 402 can be introduced into the needle being mounted.

FIG. 23B shows that in certain embodiments, a suture-loaded passer 956 can be configured such that the suture end 402 can be retained outside of the conduit 960 by a feature defined by the conduit itself. For example, a slit 958 dimensioned to wedge and retain the suture end 402 can be formed at the tip of the conduit 960. Once fluid is filled through the conduit 960 (mostly unobstructed), the suture end 402 can be pulled out of the slit 958 so as to be introduced into the needle. Since the needle's coupling sleeve likely covers the relatively small slit 958, fluid leakage between the conduit 960 and the needle is not a concern.

FIG. 23C shows that in certain embodiments, a suture-loaded passer 966 can be dry packaged and prepared for use similar to the example in FIG. 23A. In FIG. 23C, however, a plug 962 that provides similar functionality as the plug 950 of FIG. 23A can include a valve mechanism 964 that seals or allows fluid passage.

FIG. 23D shows that in certain embodiments, a suture-loaded passer 970 can include a needle assembly 976 coupled (974) to the barrel 972. The suture can be dry packaged in the passer 970 similar to that described in reference to FIG. 23A. In FIG. 23D, the free end 402 can be introduced into the needle 976 and retained therein by a retaining member 980. In certain embodiments, the retaining member 980 can be configured such that when it is in a suture-retaining position, an aperture 984 formed on the retaining member 980 allows fluid passage for filling of the chamber. When the retaining member 980 is moved (arrow 982) to release the suture end 402, the needle's interior passageway can be substantially free of the retaining member 980 so as to allow relatively free passage of fluid and the suture.

FIG. 23E shows that in certain embodiments, a suture-loaded passer 990 can be packaged with fluid 410 also preloaded into the barrel 992. As such, the plunger is shown to be in a relatively rearward position to accommodate the preloaded fluid 410.

In certain embodiments, the conduit at the tip of the barrel 992 can be substantially sealed by a removable plug 994. The plug 994 can define an extension 996 that extends into and plugs the conduit.

In certain embodiments, the suture end 402 can be secured to the plug extension 996 such that it can be pulled out of the conduit when the plug 994 is removed for use. The suture end 402 can be retained by friction in a feature formed on the extension such that the suture end 402 can be removed therefrom for insertion into the needle.

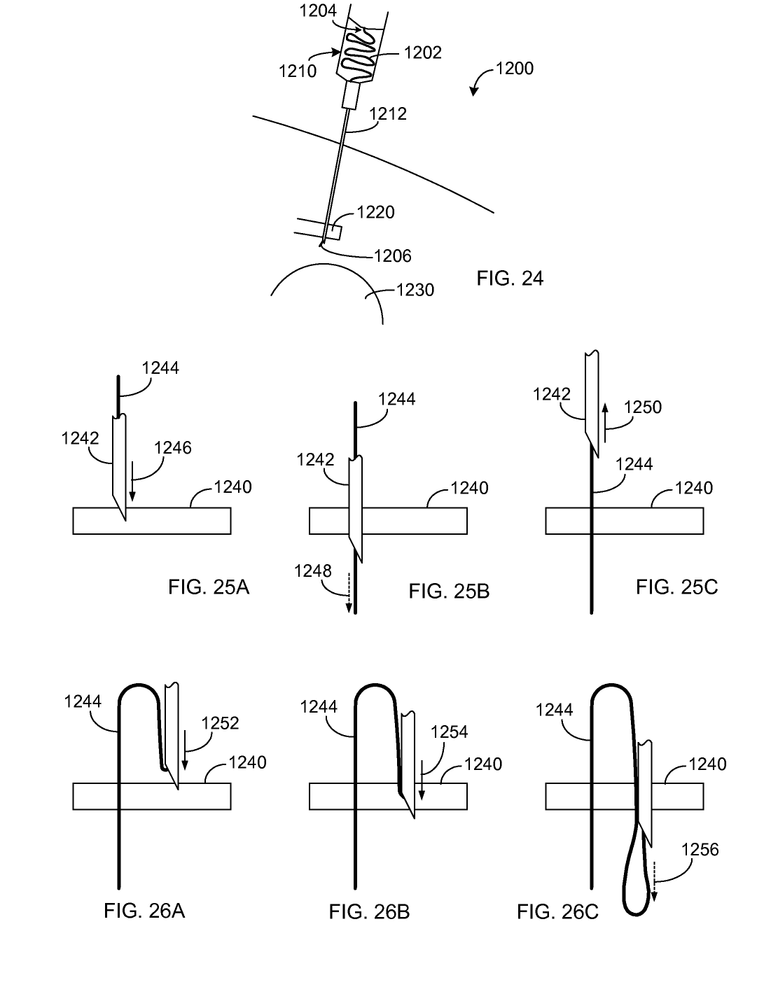

FIGS. 24-30 show non-limiting examples of suturing operations that can be performed using a suture passer having one or more features of the present disclosure. In FIG. 24, an example suture passing situation 1200 is depicted, where a suture passer 1210 having a suture 1202 can facilitate formation of a suture pattern on a tissue 1220 inside a body. As shown, one end of the suture 1202 is shown to be releasably secured (1204) to a plunger, and the other end 1206 is shown to be exiting from the tip of a needle 1212.

The needle 1212 can be introduced into the body and passed through the tissue 1220. Such passage of needle 1212 can be achieved with the suture end 1206 still inside of the needle 1212. Once the needle tip passes through the tissue 1220, fluid can be expelled so as to expel the suture end 1206 from the needle 1212. One or more further suturing steps can be performed so as to facilitate attaching of the tissue to another body part such as a bone 1230.

FIGS. 25A-25C show example steps of a basic suture passing operation that can be achieved using a suture passer having one or more features of the present disclosure. Steps depicted in FIGS. 25A and 25B can yield the example suture passing situation depicted in FIG. 24.

In FIG. 25A, a needle 1242 having a suture therein 1244 is depicted as puncturing and moving into (solid line arrow 1246) a tissue 1240. In FIG. 25B, the needle's tip has moved to the other side of the tissue 1240, and the suture 1244 is shown to be expelled from the needle 1242 via a flow of fluid (dotted line arrow 1248). In FIG. 25C, the needle 1242 is shown to be retracted (arrow 1250) from the tissue 1240, thereby leaving the passed-suture 1244 substantially in place.

In certain situations, the suture 1244 can be expelled from the suture passer (not shown) by releasing the retained end, and the two ends (one on each side of the tissue) can be collected for attaching the tissue 1240. In certain situations, the suture 1244 is not released from the suture passer, and further suturing step(s) can be performed.

For example, FIGS. 26A-26C show how the suture 1244 can be looped back for another passage through the tissue 1240. To form the loop shown in FIG. 26A, the needle can be drawn back so as to pull additional length of suture out of the passer, or some fluid can be expelled so as to expel additional suture. The needle is depicted as puncturing (arrow 1252) the tissue 1240 at a different location. As the needle moves through (arrow 1254) the tissue 1240 (FIG. 26B), the suture 1244 will have a tendency to drag behind the needle's tip by folding over an edge of the needle tip's opening. In certain embodiments, such an opening (e.g., at the trailing edge of an angled tip opening) can be rounded so as to reduce the likelihood of damage to the folded-over suture. In FIG. 26C, the needle's tip is positioned on the other side of the tissue 1240 so as to allow expelling (arrow 1256) of additional suture 1244 from the needle.

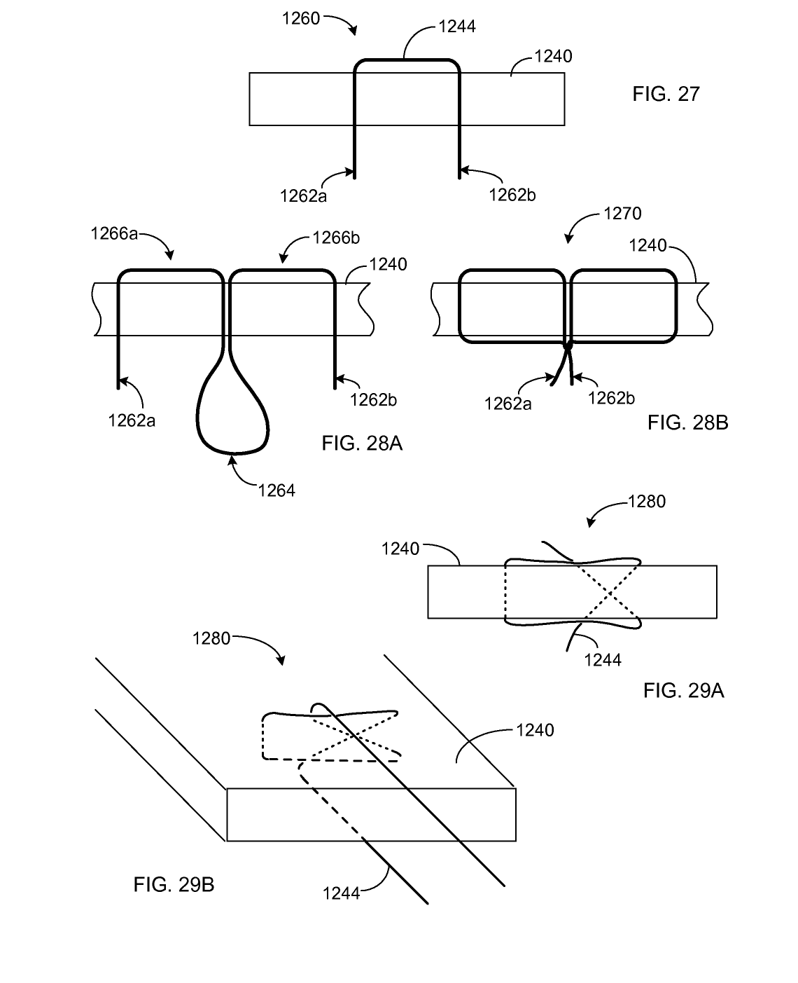

FIGS. 27 and 28 show examples of how the suturing steps shown in FIGS. 25 and 26 can form mattress stitching patterns. In FIG. 27, a single mattress suture pattern 1260 can be achieved by expelling the suture end from the suture passer (via the needle) in the example step of FIG. 26C, so as to yield two ends 1262a and 1262b of the suture 1244 on the same side of the tissue 1240. Both ends 1262 of the suture 1244 can then be pulled so as to tighten the loop on the other side of the tissue 1240, and the ends 1262 can be secured to, for example, an anchor nearby (e.g., an anchor secured to the bone 1230 in FIG. 24).

To form a double mattress suture pattern of FIG. 28B, the needle in the example step of FIG. 26C (where a first of the double mattress pattern has been formed on the entering side) can be withdrawn to the entering side of the tissue 1240 without expelling the suture 1244. Such a needle motion results in a loop 1264 being formed. A second portion of the double mattress pattern can be achieved in a manner similar to that described in reference to FIGS. 26A-26C, followed by expelling of the suture from the passer.

As shown in FIG. 28A, the resulting suture configuration includes the first and second portions 1266a and 1266b of the double mattress pattern, with the loop 1264 on the other side. As shown in FIG. 28B, both ends 1262a and 1262b of the suture can be threaded through the loop 1264 and pulled so as to yield a tightened double mattress suture pattern 1270.

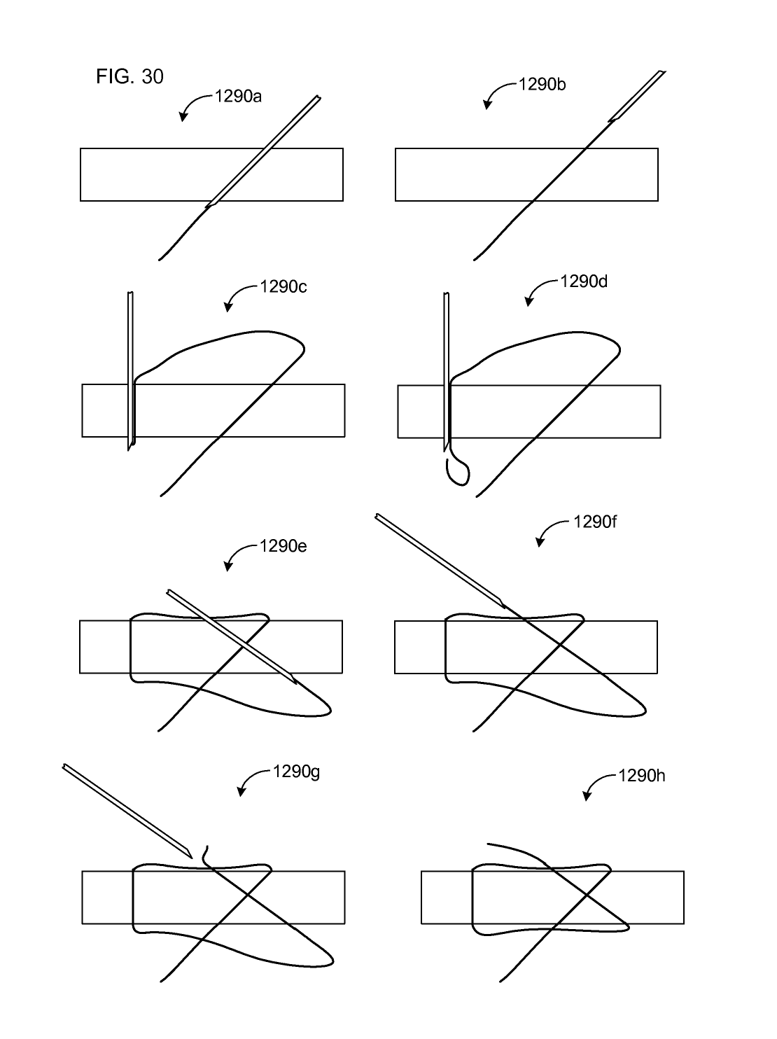

In certain situations, more complex suture patterns can be achieved. For example, FIGS. 29A and 29B show perspective and end views of a tissue 1240 where a modified Mason-Allen suture pattern 1280 is formed by the thread. FIG. 30 shows an example sequence of suture passing steps that can be performed so as to achieve the modified Mason-Allen suture pattern 1280 of FIGS. 29A and 29B.

For the purpose of description of FIG. 30, the side from which the needle enters the tissue is designated as the first side; and the other side the second side. In step 1290a, the needle is shown to have passed the first end of the suture from the first side to the second side at an angle. In step 1290b, the needle can be withdrawn back to the first side so as to yield the suture being at an angle through the tissue. In step 1290c, the needle is moved laterally and passed to the second side with the suture folded over its tip. In step 1290d, the second end of the suture is shown to be expelled from the suture passer via the needle into the second side.

In certain situations, another suture passer's needle having a suture-retrieving suture (e.g., by having an appropriately sized loop structure at the free end) can be passed through the tissue along a direction that generally crosses the angled suture from step 1290b. The second free end of the suture from step 1290d can be threaded through such a retrieving loop (not shown). In step 1290e, the second end of the suture is depicted as being sucked back into the retrieving suture passer. The needle associated with the retrieving suture passer and the suture-retrieving suture can be selected to allow such passage of suture folded over the retrieving loop.

In step 1290f, the retrieving needle is shown to be withdrawn back to the first side, such that the retrieved second end portion of the suture generally crosses the angled suture orientation of step 1290b. In step 1290g, the retrieved second end of the suture is shown to be expelled from the retrieving needle by expelling motion of fluid. In certain situations, the second end of the suture may need to be manipulated out of the retrieving loop. In step 1290h, the first end of the suture on the second side and the second end of the suture on the first side can be collected and pulled so as to tighten the suture pattern. Both ends of the suture can then be secured to, for example, a suture anchor.

In certain situations, it may be desirable to manipulate the suture passer to some degree when during various suture passing and/or retrieving operations. To facilitate such manipulations, additional ergonomic features can be provided for the suture passer device.

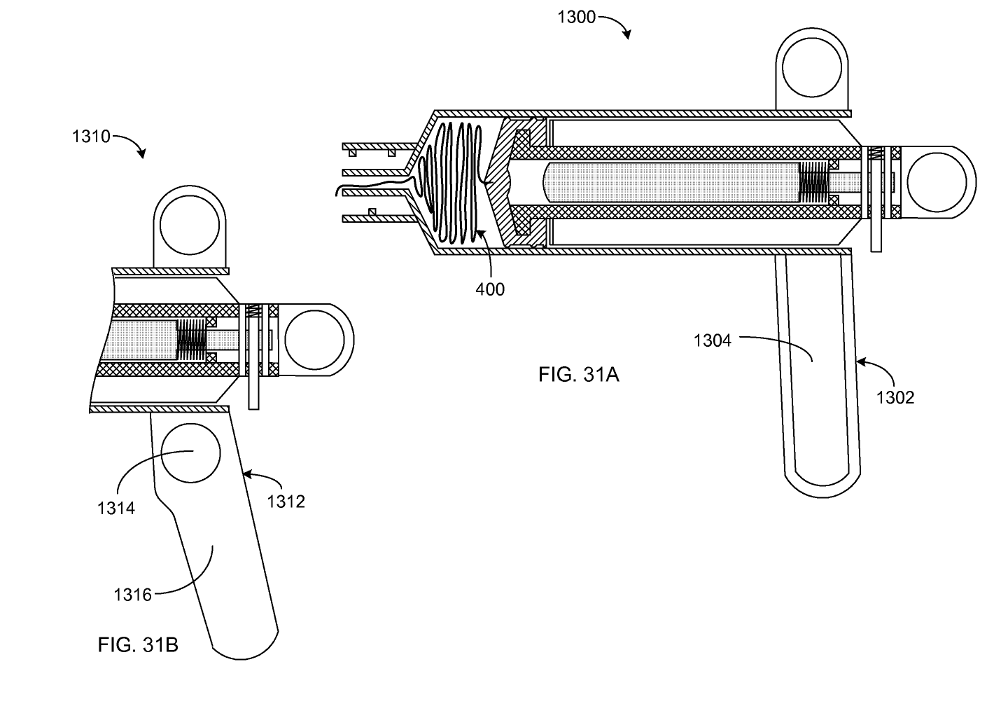

For example, FIG. 31A shows an example suture passer 1300 having a loaded suture 400 (and one end releasably secured). As described herein, such a device can be provided with one or more ergonomic features that can enhance the operating performance of the device. For example, the example suture passer in FIGS. 13A and 13B has support rings for the thumb and two fingers. In the example suture passer 1300, one of the finger support rings can be replaced with a larger support member 1302 that defines an opening 1304. The support member 1302 and the opening 1304 can be dimensioned so as to allow a pistol-like grip by either inserting three fingers (middle, ring, and little) through the opening 1304, holding the three fingers outside the opening 1304, or some combination thereof. In certain situations, such additional grip of the suture passer device 1300 may provide additional manipulation performance.

FIG. 31B shows a variation to the example suture passer device of FIG. 31A. A suture passer device 1310 can have a similar grip feature 1312 that defines a finger support hole 1314 and a grip portion 1316 that can be gripped by two or more fingers (e.g., ring, little).

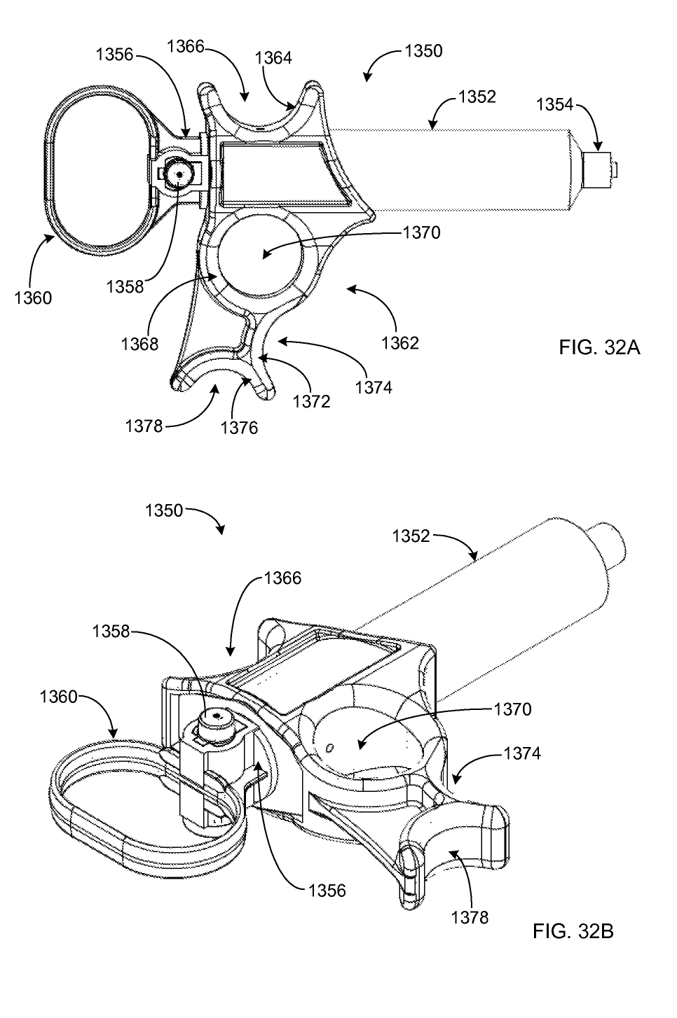

FIGS. 32A and 32B show another example of a suture passer device 1350 having ergonomic features that can enhance the operating performance of the device. FIG. 32A shows a side view, and FIG. 32B shows an isometric view of the example device 1350.

The suture passer device 1350 is shown to include a barrel 1352 and a conduit tip 1354 that can be configured similar to one or more barrel configurations described herein. A plunger assembly 1356 is shown to be in a forward position (relative to the barrel 1352) for the purpose of description. The plunger 1356 can have a suture release mechanism (not shown), and a suture (not shown) can be disposed within the barrel 1352, in manners similar to those described herein. In FIGS. 32A and 32B, the example plunger 1356 is shown to include an actuator triggering button 1358 that can function similar to the example described herein in reference to FIGS. 13A-13C. It will be understood, however, that other actuator mechanisms can be implemented with the ergonomic features shown in FIGS. 32A and 32B.

In certain embodiments, ergonomic features can include a thumb-retaining ring 1360 disposed at the end of the plunger 1356 in a manner similar to one or more examples described herein. Ergonomic features can further include a grip 1362 having a plurality of features that accommodate various fingers of the user. For example, an index finger can typically be positioned in a recess 1366 defined by a curved portion 1364. A middle finger can typically be positioned in a finger hole 1370 defined by a curved portion 1368.

Depending on the user's hand size and/or preference, ring finger and little finger can be positioned in one or more additional recesses defined further away from the barrel 1352. For example, the ring finger can be positioned in a recess 1374 defined by a curved portion 1372, and the little finger can be positioned in a recess 1378 defined by a curved portion 1376.

In certain embodiments, the curved portions 1364 and 1376 of the grip 1362 can be dimensioned such that their corresponding recesses 1366 and 1378 are partially open. In use, fingers that occupy such partially open recesses (1366, 1378) typically will not encounter a situation where fully closed recesses (such as the aperture 1370) are needed. For example, an index finger occupying the recess 1366 and a little finger occupying the recess 1378 are likely spread out from each other to some degree, thereby having tendencies to move towards each other so as to engage inner sides (towards the barrel 1352) of the curved portions 1364 and 1376. In certain embodiments, providing such partially open recesses for outer-most fingers (relative to the barrel 1352) can desirably reduce the overall lateral dimension of the grip 1362 (when compared to a configuration where the recesses 1366 and/or 1378 are fully enclosed). Further, such open recesses can also provide easier positioning and removal of the fingers to and from the grip 1362.

Preferably, various curved portions of the grip 1362 are dimensioned to be user-friendly (e.g., rounded and dimensioned appropriately). In certain embodiments, the grip 1362 can be formed as an integral part of the barrel 1352 (e.g., by injection molding). In certain embodiments, the grip 1362 can be a separate piece or assembly that can be secured to the barrel 1352 via a number of ways.

Other ergonomic features and configurations are possible.

Although the above-disclosed embodiments have shown, described, and pointed out the fundamental novel features of the invention as applied to the above-disclosed embodiments, it should be understood that various omissions, substitutions, and changes in the form of the detail of the devices, systems, and/or methods shown may be made by those skilled in the art without departing from the scope of the invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A syringe assembly comprising:
a barrel that defines a chamber and a conduit having a reduced dimension;
a plunger having a suture retaining mechanism; and
a suture substantially positioned in said chamber, one end of said suture held releasably by said suture retaining mechanism.

2. The syringe assembly of claim 1, wherein said barrel includes a needle-coupling mechanism at or about said conduit so as to allow flow of liquid through a needle coupled to said barrel as said liquid is expelled from or drawn into said chamber by action of said plunger.

3. The syringe assembly of claim 2, wherein other end of said suture is positioned at or about said conduit so as to allow said other end of said suture to enter said needle.

4. The syringe assembly of claim 3, further comprising a conduit feature disposed on or about said conduit and configured to inhibit said other end of said suture from being drawn into said chamber.

5. The syringe assembly of claim 1, wherein said suture retaining mechanism comprises a deformable tip of said plunger, said deformable tip having a feature formed at its front, said front facing said chamber, said feature configured to retain said one end of said suture in a closed configuration and release said suture in an open configuration.

6. The syringe assembly of claim 5, wherein said plunger further comprises an actuator mechanism that transmits a force to said deformable tip so as to facilitate transition of said feature from said closed configuration to said open configuration.

7. The syringe assembly of claim 6, wherein said actuator mechanism comprises an actuator rod that provides a pushing force to said deformable tip from rear of said deformable tip so as to deform an area at or about said feature sufficiently to yield said transition.

8. The syringe assembly of claim 7, wherein said actuator mechanism further comprises a hollow rod coupled to said deformable tip, said hollow rod defining a bore dimensioned to receive said actuator rod and allow axial motion of said actuator rod relative to said hollow rod so as to provide said pushing force to said deformable tip.

9. The syringe assembly of claim 5, wherein said feature comprises one or more slits formed on said deformable tip and dimensioned to squeeze and retain said one end of said suture in said closed configuration and spread and release said suture in said open configuration.

10. The syringe assembly of claim 9, wherein said slit is dimensioned such that said rubber tip retains its fluid sealing property when said slit is in either said closed or open configuration.

11. The syringe assembly of claim 1, wherein said suture is stored in a selected configuration relative to a feature associated with said plunger.

12. The syringe assembly of claim 11, wherein said feature comprises a plunger tip having a reduced diameter portion such that said suture is wound about said reduced diameter portion.

13. The syringe assembly of claim 11, wherein said feature comprises a recess defined by a tip of said plunger such that said suture is in a coiled configuration substantially within said recess.

14. The syringe assembly of claim 1, wherein said suture retaining mechanism comprises a jaw assembly disposed at a tip of said plunger, said jaw assembly configured to retain said suture in a closed position, and release said suture in an open position, opening of said jaw assembly induced by an interaction of said jaw assembly with a feature associated with said barrel.

15. A kit comprising:
said syringe assembly of claim 1; and
a sterile packaging for said syringe assembly.

16. The kit of claim 15, wherein said syringe assembly is substantially dry when in said sterile packaging.

17. The kit of claim 15, wherein said syringe assembly further includes a pre-loaded fluid in said chamber.

18. A device, comprising:
a barrel having proximal and distal ends, and defining an inner volume having a first cross-sectional area and extending axially between said proximal and distal ends, said distal end of said barrel defining an opening having a second cross-sectional area that is less than said first cross-sectional area, said opening configured to allow expelling and drawing of fluid from and into said inner volume through said opening;
a plunger having proximal and distal ends, said distal end of said plunger configured to fit in said inner volume and be movable axially so as to decrease and increase said inner volume to thereby facilitate said expelling and drawing of said fluid; and
a suture holding mechanism disposed on or about said distal end of said plunger and configured to releasably hold an end of a suture.

19. The device of claim 18, further comprising an actuator mechanism configured to release said end of said suture from said suture holding mechanism.

20. The device of claim 19, wherein said actuator mechanism comprises one or more parts disposed within said plunger, said one or more parts movable relative to said distal end of said plunger.

21. The device of claim 19, wherein said actuator mechanism induces said suture release by an interaction between said plunger and said barrel.

22. The device of claim 18, wherein said suture holding mechanism comprises one or more features formed on a tip disposed at said distal end of said plunger.

23. The device of claim 18, wherein said suture holding mechanism comprises one or more parts secured to a tip disposed at said distal end of said plunger.

24. A method, for performing a suturing operation, comprising:
(a) passing a needle from a first side to a second side of a tissue;
(b) expelling a first end of a suture from said needle such that said first end is at said second side;
(c) performing one or more cycles of:
(i) withdrawing said needle back to said first side such that a part of suture that came out of said needle just prior to said withdrawing remains substantially at said second side;
(ii) moving said needle to a different location on said tissue; and
(iii) passing said needle from said first side to said second side at said different location such that a part of said suture outside of said needle follows said needle from said first side to said second side; and
(d) expelling a second end of said suture from said needle such that said second end is at said second side.

25. The method of claim 24, wherein said expelling of said first and second ends of said suture are achieved by expelling of fluid through said needle.

26. The method of claim 25, wherein said expelling of fluid is achieved by a suture passer coupled to said needle, said suture passer having a chamber defined by a barrel and a plunger, said fluid being expelled from said chamber, and at least some of said suture being in said chamber prior to said step (d) such that said second end is releasably retained by said plunger.

27. The method of claim 24, wherein said step (c) is performed once so as to form a single mattress suture pattern.

28. The method of claim 24, wherein said step (c) is performed twice so as to form a double mattress suture pattern by passing said first and second ends at second side of said tissue through an intermediate loop of suture formed on second side of said tissue and tightening said first and second ends.
